(19) United States
(12) Patent Application Publication
(10) Pub. No.: US 2025/0320269 A1
(43) Pub. Date: Oct. 16, 2025

(54) CHIMERIC ANTIGEN RECEPTORS TARGETING B7-H3 (CD276) AND ASSOCIATED METHODS

(71) Applicant: Albert Einstein College of Medicine, Bronx, NY (US)

(72) Inventor: Xingxing ZANG, New York, NY (US)

(21) Appl. No.: 17/757,249

(22) PCT Filed: Dec. 18, 2020
(86) PCT No.: PCT/US2020/066002

§ 371 Date: Jun. 13, 2022

Related U.S. Application Data
(60) Provisional application No. 62/949,717, filed on Dec. 18, 2019.

(51) Int. Cl. C07K 14/725; C07K 14/705; C07K 14/71; C07K 16/28

(52) U.S. Cl. CPC C07K 14/7051; C07K 14/70517; C07K 14/70521; C07K 14/71; C07K 16/2827; C07K 2317/24; C07K 2319/03

(57) ABSTRACT

In various embodiments, the present disclosure provides chimeric antigen receptors (CAR)s which bind to B7-H3. The B7-H3 CARs comprise an extracellular region comprising a binding domain that specifically binds to at least a portion of B7-H3, a transmembrane region, and an intracellular region comprising an effector domain or a portion or variant thereof and a costimulatory domain or a portion or variant thereof. Recombinant host cells expressing the CARs are also provided, as well as compositions and methods comprising the same.

### CROSS-REFERENCE TO RELATED APPLICATIONS

The present application claims priority to U.S. Provisional Application No. 62/949,717, filed Dec. 18, 2019, which is incorporated herein by reference in its entirety, including the drawings.

### STATEMENT REGARDING FEDERAL FUNDING

This invention was made with government support under CA175495 and DK100525 awarded by the National Institutes of Health. The government has certain rights in this invention.

### SEQUENCE LISTING

This application contains a Sequence Listing, which was submitted in ASCII format via EFS-Web, and is hereby incorporated by reference in its entirety. The ASCII copy, created on Dec. 18, 2020, is name Sequence_Listing.txt and is 72 KB in size.

### BACKGROUND

Adoptive transfer of chimeric antigen receptor (CAR) modified T cells or NK cells is a potent antigen-specific therapy for treating diseases, such as human malignancies. CAR expressing T cells or NK cells target a specific antigen expressed on a malignant cell's surface. Since CAR T or NK cell-based therapeutic strategies target a specific antigen that is already expressed on a cell's surface, issues associated with tumor escape mechanisms involving major histocompatibility complexes can be overcome.

One cell surface antigen expressed by malignant cells is B7-H3 (also known as CD276), a Type I transmembrane protein that belongs to the B7 family. The extracellular portion of B7-H3 is composed of four or two immunoglobulin domains, either two variable domains and two constant domains (e.g., IgV-IgC-IgV-IgC) or one variable domain and one constant domain (e.g., IgV-IgC). The FG loop of the IgV domain(s) is involved in B7-H3-mediated T cell suppression (Vigdorovich et al., 2013).

B7-H3 is an immune checkpoint protein used by cancer cells to inhibit immune cell functions and is overexpressed in human malignancies (Picarda et al., 2006; Zang et al., 2007) including prostate cancer (Zang et al., 2007), liver cancer (Sun et al., 2012), melanoma (Wang et al., 2013), leukemia (Hu et al., 2015), breast cancer (Sun et al., 2014), ovarian cancer (Zang et al., 2010), pancreatic cancer (Chen et al., 2014), colorectal cancer (Ingebrigtsen et al., 2014), lung cancer (Sun et al., 2006), bladder cancer (Xylinas et al., 2014), renal cancer (Qin et al., 2013), brain cancer (Baral et al., 2014), osteosarcoma (Wang et al., 2013), and other cancers. High expression of B7-H3 often correlates with poor prognosis and an unfavorable clinical outcome (Picarda et al., 2006; Zang et al., 2007).

New strategies for treating human malignancies are needed, especially those involving CAR modified T cells or NK cells targeting B7-H3.

### SUMMARY

In some aspects, the present disclosure provides a chimeric antigen receptor (CAR), comprising (a) an extracellular region comprising a binding domain that specifically binds to at least a portion of B7-H3, (b) a transmembrane region; and (c) an intracellular region comprising an effector domain or a portion or variant thereof and a costimulatory domain or a portion or variant thereof.

In some embodiments, the binding domain is an scFv.

In some embodiments, the scFv includes at least a V_{L} chain of an antibody which binds to B7-H3 or a portion or variant thereof and a V_{H} chain of an antibody which binds to B7-H3 or a portion or variant thereof. In some embodiments, the V_{L} chain includes at least complement determining regions (CDR) 1, CDR 2, and CDR 3, and at least framework regions (FR) 1, FR 2, FR 3, and FR 4. In some embodiments, the V_{H} chain includes at least CDR 1, CDR 2, and CDR 3, and at least FR 1, FR 2, FR 3, and FR 4. In some embodiments, the V_{L} chain comprises an amino acid sequence having at least 75% identity to the amino acid sequences shown in SEQ ID NO: 8, 10, 12, 14, 16, or 18. In some embodiments, the V_{L} chain comprises an amino acid sequence selected from the group consisting of SEQ ID NO: 8, 10, 12, 14, 16, or 18. In some embodiments, the V_{H} chain comprises an amino acid sequence having at least 75% identity to the amino acid sequences shown in SEQ ID NO: 7, 9, 11, 13, 15, or 17. In some embodiments, the V_{H} chain comprises an amino acid sequence selected from the group consisting of SEQ ID NO: 7, 9, 11, 13, 15, or 17.

In some embodiments, the extracellular region further comprises a linker or a portion or variant thereof. In some embodiments, the linker is a glycine-serine linker. In some embodiments, the glycine-serine linker comprises GlyₓSer_{y} or about two to about ten repeats of GlyₓSer_{y}. In some embodiments, the linker is disposed between the V_{L} and V_{H} domains.

In some embodiments, the extracellular region further comprises a leader or a portion or variant thereof. In some embodiments, the leader is disposed N-terminal of the V_{L} domain. In some embodiments, the leader is a V_{K} domain of IgG1 or a portion or variant thereof.

In some embodiments, the transmembrane region comprises or is a combination of (i) a CD8α hinge or a portion or variant thereof and (ii) CD8α transmembrane region or a portion or variant thereof. In some embodiments, the transmembrane region comprises an amino acid sequence having at least 75% identity to the amino acid sequence shown in SEQ ID NO: 1.

In some embodiments, the effector domain or portion or variant thereof is CD3ζ or a portion or variant thereof. In some embodiments, the effector domain comprises an amino acid sequence having at least 75% identity to the amino acid sequence shown in SEQ ID NO: 4.

In some embodiments, the costimulatory domain or portion or variant thereof is a CD28 costimulatory domain or a portion or variant thereof, a 4-1 BB costimulatory domain or a portion or variant thereof, or a combination thereof. In some embodiments, the CD28 costimulatory domain comprises an amino acid sequence having at least 75% identity to the amino acid sequence shown in SEQ ID NO: 2. In some embodiments, the 4-1BB costimulatory domain comprises an amino acid sequence having at least 75% identity to the amino acid sequence shown in SEQ ID NO: 3. In some embodiments, the costimulatory domain comprises a CD28 costimulatory domain or a portion or variant thereof and the effector domain comprises CD3ζ or a portion or variant thereof. In some embodiments, the costimulatory domain comprises an amino acid sequence having at least 75% identity to the amino acid sequence shown in SEQ ID NO: 2 and the effector domain comprises an amino acid sequence having at least 75% identity to the amino acid sequence shown in SEQ ID NO: 4. In some embodiments, the costimulatory domain comprises a 4-1 BB costimulatory domain or a portion or variant thereof and the effector domain comprises CD3ζ or a portion or variant thereof. In some embodiments, the costimulatory domain comprises an amino acid sequence having at least 75% identity to the amino acid sequence shown in SEQ ID NO: 3 and the effector domain comprises an amino acid sequence having at least 75% identity to the amino acid sequence shown in SEQ ID NO: 4. In some embodiments, the costimulatory domain comprises a CD28 costimulatory domain or a portion or variant thereof and a 4-1 BB costimulatory domain or a portion or variant thereof, and the effector domain comprises CD3ζ or a portion or variant thereof. In some embodiments, the costimulatory domain comprises an amino acid sequence having at least 75% identity to the amino acid sequence shown in SEQ ID NO: 2 and an amino acid sequence having at least 75% identity to the amino acid sequence shown in SEQ ID NO: 3, and the effector domain comprises an amino acid sequence having at least 75% identity to the amino acid sequence shown in SEQ ID NO: 4.

In some embodiments, the binding domain is chimeric, human, or humanized.

In some aspects, the present disclosure provides an isolated polynucleotide, encoding any of the CARs described herein.

In some aspects, the present disclosure provides an expression vector, comprising any of the isolated polynucleotides described herein operably linked to an expression control sequence. In some embodiments, the expression control sequence is a promoter.

In some embodiments, the expression vector further comprises an isolated polynucleotide encoding a self-cleaving peptide. In some embodiments, the self-cleaving peptide is a 2A self-cleaving peptide. In some embodiments, the 2A self-cleaving peptide is a P2A peptide. In some embodiments, the isolated polynucleotide encoding the self-cleaving peptide is 3′ of the polynucleotide encoding the CAR. In some embodiments, the isolated polynucleotide encoding the self-cleaving peptide is 5′ of the isolated polynucleotide encoding the marker polypeptide.

In some embodiments, the expression vector further comprises an isolated polynucleotide encoding a transduction marker polypeptide. In some embodiments, the transduction marker polypeptide is a truncated form of epidermal growth factor receptor (EGFRt) or a portion or variant thereof or GFP or a portion or variant thereof. In some embodiments, the EGFRt or a portion or variant thereof comprises an amino acid sequence having at least 75% identity to the amino acid sequence shown in SEQ ID NO: 6.

In some embodiments, the expression vector is capable of delivering the isolated polynucleotides to a host cell.

In some embodiments, the vector is a viral vector.

In some aspects, the present disclosure provides a host cell expressing any of the CARs of the present disclosure, and/or comprising any of the isolated polynucleotides of the present disclosure, and/or comprising the expression vector of the present disclosure.

In some embodiments, the host cell is a T cell, a natural killer (NK) cell, a yδT cell, an NKT cell, a B cell, a macrophage, a dendritic cell, or an innate lymphoid cell. In some embodiments, the T cell is a CD4+ T cell, a CD8+ T cell, a CD4⁻ CD8⁻ double negative T cell, a yδT cell, an NKT cell, or any combination thereof. In some embodiments, the T cell is a naïve T cell, a central memory T cell, a stem cell memory T cell, an effector memory T cell, a yδT cell, an NKT cell, or any combination thereof.

In some embodiments, the host cell further expresses a transduction marker at its cell surface. In some embodiments, the transduction marker is a truncated form of epidermal growth factor receptor (EGFRt) or a portion or variant thereof or GFP or a portion or variant thereof. In some embodiments, the EGFRt or a portion or variant thereof comprises an amino acid sequence having at least 75% identity to the amino acid sequence shown in SEQ ID NO: 6.

In some aspects, the present disclosure provides a composition, comprising a host cell of the present disclosure and a pharmaceutically acceptable carrier, pharmaceutically acceptable excipient, and/or pharmaceutically acceptable diluent.

In some aspects, the present disclosure provides a method of treating a disease or condition in a subject, the method comprising administering to the subject an effective amount of the host cell of the present disclosure, wherein the disease or condition is diagnosed in the subject by the presence of B7-H3.

In some embodiments, the disease or condition is a malignancy. In some embodiments, the malignancy is a cancer. In some embodiments, the cancer is selected from the group consisting of prostate cancer, liver cancer, melanoma, leukemia, breast cancer, ovarian cancer, pancreatic cancer, colorectal cancer, lung cancer, bladder cancer, renal cancer, brain cancer, rectal cancer, cancer of the small intestine, cancer of the esophagus, bone cancer, skin cancer, cancer of the head or neck, uterine cancer, cancer of the anal region, stomach cancer, testicular cancer, cancer of the fallopian tubes, cancer of the endometrium, cancer of the cervix, cancer of the vagina, cancer of the vulva, cancer of the endocrine system, cancer of the thyroid gland, cancer of the parathyroid gland, cancer of the adrenal gland, sarcoma of soft tissue, cancer of the urethra, cancer of the penis, solid tumors of childhood, neoplasm of the central nervous system (CNS), primary CNS lymphoma, tumor angiogenesis, spinal axis tumor, squamous cell cancer, osteosarcoma, Kaposi's sarcoma, epidermoid cancer, environmentally induced cancers, combinations of the cancers, and metastatic lesions of the cancers. In some embodiments, the cancer comprises a solid tumor.

In some embodiments, the cancer is a human hematologic malignancy. For example, in certain embodiments the human hematologic malignancy may be selected from myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplatic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, Waldenström macroglobulinemia, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some aspects, the present disclosure provides a method of eliciting an immune response against B7-H3 that requires binding of any of the CARs of the present disclosure to B7-H3, the method comprising administering to a subject having a disease or condition diagnosed by expression of B7-H3 an effective amount of any of the host cells of the present disclosure.

In some embodiments, methods of the present disclosure further comprise administering at least one-unit dose of any of the host cells of the present disclosure to the subject. In some embodiments, a second unit dose is administered to the subject about two, three, four, five, six, seven, eight, nine, ten, eleven, twelve, thirteen, fourteen, fifteen, sixteen or more weeks after a first unit dose is administered to the subject. In some embodiments, the at least one-unit dose, first unit dose, and/or second unit dose comprises about 10⁵ cells/m² to about 10¹¹ cells/m², inclusive.

In some aspects, the present disclosure provides a use of any of the CARs of the present disclosure, or any of the expression vector of the present disclosure, or any of the host cells of the present disclosure, in the manufacture of a medicament for the treatment of a disease or condition diagnosed by expression of B7-H3 on at least one proliferative cell.

In some embodiments, the at least one proliferative cell is a malignant cell. In some embodiments, the malignant cell is a cancer cell.

These and other embodiments of the present disclosure will be disclosed in further detail herein below.

### BRIEF DESCRIPTION OF THE DRAWINGS

FIG. 1A is a schematic representation of a B7-H3 chimeric antigen receptor (CAR) in accordance with an embodiment of the present disclosure.

FIG. 1B is a schematic representation of a tagged variant of the B7-H3 CAR of FIG. 1A in accordance with an embodiment of the present disclosure.

FIG. 1C is a schematic representation of another tagged variant of the B7-H3 CAR of FIG. 1A in accordance with an embodiment of the present disclosure.

FIG. 2A is a schematic representation of another B7-H3 CAR in accordance with an embodiment of the present disclosure.

FIG. 2B is a schematic representation of a tagged variant of the B7-H3 CAR of FIG. 2A in accordance with an embodiment of the present disclosure.

FIG. 2C is a schematic representation of another tagged variant of the B7-H3 CAR of FIG. 2A in accordance with an embodiment of the present disclosure.

FIG. 3A is a schematic representation of yet another B7-H3 CAR in accordance with an embodiment of the present disclosure.

FIG. 3B is a schematic representation of a tagged variant of the B7-H3 CAR of FIG. 3A in accordance with an embodiment of the present disclosure.

FIG. 3C is a schematic representation of another tagged variant of the B7-H3 CAR of FIG. 3A in accordance with an embodiment of the present disclosure.

FIG. 4A is a schematic representation of two B7-H3 CARs in accordance with an embodiment of the present disclosure.

FIG. 4B depicts a representative fluorescence assisted cell sorting (FACS) plot for T cells expressing one of the B7-H3 CARs of FIG. 4A.

FIG. 5 depicts a representative graph showing that T cells expressing a B7-H3 CAR in accordance with an embodiment of the present disclosure kill at least two different types of tumor cells.

FIG. 6A is a schematic representation of different anti-B7-H3 CAR-T vectors generated from anti-B7-H3 mAbs.

FIG. 6B is FACS showing that anti-B7-H3 CAR-T vector transduced human primary T cells expressed EGFR and recognized B7-H3 antigen. Non-transduced human primary T cells did not express EGFR and did not recognize B7-H3 antigen.

### DETAILED DESCRIPTION

The present disclosure provides chimeric antigen receptors (CARs) which bind to B7-H3, cells expressing these CARs, and methods of using these CARs and cells expressing the same.

When expressed by a cell and bound to B7-H3 expressed by a target cell, the B7-H3 CARs provided herein induce initiation, propagation, and/or magnification of a molecular signal in the cell, such as cytotoxicity, proliferation, and/or survival. Exemplary CARs of the present disclosure comprise (a) an extracellular region comprising a binding domain (e.g., an scFv) that specifically binds to B7-H3; (b) a transmembrane region; and (c) an intracellular region comprising an effector domain or a portion or variant thereof, and a costimulatory domain or a portion or variant thereof.

The B7-H3 CARs of the present disclosure are useful in cellular immunotherapies (e.g., T cells, NK cells, yδT cells, NKT cells, B cells, macrophages, dendritic cells, and innate lymphoid cells) for treating a disease or condition associated with B7-H3 expression, such as, a malignancy. In some embodiments, when administered to a subject having target cells (e.g., malignant cells) that express B7-H3, cells expressing B7-H3 CARs of the present disclosure reduce and/or suppress growth, area, volume, and/or spread of the malignant cells, eliminate (e.g., kill) malignant cells, and/or increase survival of the subject to a greater degree and/or for a longer period of time than cells that do not comprise a B7-H3 CAR of the present disclosure.

In some embodiments, a T cell, a NK cell, a yδT cell, an NKT cell, a B cell, a macrophage, a dendritic cell, or an innate lymphoid cell expressing a B7-H3 CAR described herein demonstrates increased and/or sustained cell signaling, such as cytokine production and/or release, phosphorylation of one or more proteins associated with a T cell response to antigen-binding, and/or activity, such as mobilization of intracellular calcium, cytotoxic activity, secretion of a cytokine, proliferation, and/or activation following stimulation. One or more of these effects occurring in response to B7-H3 binding is improved relative to a T cell and/or a NK cell that does not express a B7-H3 CAR of the present disclosure.

The following description of the present disclosure is merely intended to illustrate various embodiments of the present disclosure. As such, the specific modifications discussed herein are not to be construed as limitations on the scope of the present disclosure. It will be apparent to one skilled in the art that various equivalents, changes, and modifications may be made without departing from the scope of the present disclosure, and it is understood that such equivalent embodiments are to be included herein.

Reference throughout this specification to “one example,” “an example,” “one embodiment,” “an embodiment,” “one aspect,” or “an aspect” means that a particular feature, structure, or characteristic described in connection with the example is included in at least one example of the present disclosure. Thus, the occurrences of the phrases “in one example,” “in an example,” “one embodiment,” “an embodiment,” “one aspect,” or “an aspect” in various places throughout this specification are not necessarily all referring to the same example, embodiment, and/or aspect.

The headings provided herein are for convenience only and are not intended to limit or interpret the scope or meaning of the present disclosure.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense that one having skill in the art would understand the convention (e.g., “a system having at least one of A, B, or C” would include but not be limited to systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting.

The terms “peptide,” “polypeptide,” and “protein” are used interchangeably to refer to a polymer of amino acid residues, and are not limited to a minimum length, though a number of amino acid residues may be specified. Polypeptides may include amino acid residues including natural and/or non-natural amino acid residues. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified. Amino acid analogs refer to compounds that have the same basic chemical structure as a naturally occurring amino acid. Such analogs have modified R groups or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L and nor leucine (NIe).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (GIn or Q); Group 4: Arginine (Arg or R), Lysine (Lys or K), Histidine (His or H); Group 5: Isoleucine (IIe or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and IIe. Other conservative substitutions groups include: sulfur-containing: Met and Cysteine (Cys or C); acidic: Asp, Glu, Asn, and GIn; small aliphatic, nonpolar or slightly polar residues: Ala, Ser, Thr, Pro, and Gly; polar, negatively charged residues and their amides: Asp, Asn, Glu, and GIn; polar, positively charged residues: His, Arg, and Lys; large aliphatic, nonpolar residues: Met, Leu, IIe, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine, and guanine, and pyrimidine bases including uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

As used herein, the terms “homologous,” “homology,” or “percent homology” when used herein to describe to a nucleic acid sequence, relative to a reference sequence, can be determined using the formula described by Karlin & Altschul 1990, modified as in Karlin & Altschul 1993. Percent homology of sequences can be determined using the most recent version of BLAST, as of the filing date of this application.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “mutation” refers to a change in the sequence of a polynucleotide molecule or polypeptide molecule as compared to a reference or wild-type polynucleotide molecule or polypeptide molecule, respectively. A mutation can result in several different types of change in sequence, including substitution, insertion or deletion of nucleotide(s) or amino acid(s).

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed; or one or more amino acids are mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. Here, “plasmid,” “expression plasmid,” “virus” and “vector” are often used interchangeably.

The term “introduced” in the context of inserting a nucleic acid molecule into a cell, means “transfection,” or “transformation” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, CARs or enzymes, or other nucleic acid molecule additions, deletions, substitutions or other functional disruption of a cell's genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related or unrelated to, biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4⁺ cells while depleting CD8⁺ cells, or enriching for CD8⁺ cells while depleting CD4⁺ cells, or combinations thereof.

“Chimeric antigen receptor” (CAR) refers to a CAR of the present disclosure engineered to contain two or more naturally occurring (or engineered) amino acid sequences linked together in a way that does not occur naturally or does not occur naturally in a host cell, which CAR can function as a receptor when present on a surface of a cell. CARs of the present disclosure include an extracellular portion comprising an antigen-binding domain, such as one obtained or derived from an immunoglobulin, such as an scFv derived from an antibody linked to a transmembrane region and one or more intracellular signaling domains (optionally containing co-stimulatory domain(s)) (see, e.g., Sadelain et al., 2013; see also Harris & Kranz, 2016; Stone et al., 2014).

The term “variable region” or “variable domain” refers to an antibody heavy or light chain, that is involved in binding to antigen. Variable domains of antibody heavy (V_{H}) and light (V_{L}) chains each generally comprise four generally conserved framework regions (FRs) and three CDRs. Framework regions separate CDRs, and CDRs are situated between framework regions.

The terms “complementarity determining region,” and “CDR,” are synonymous with “hypervariable region” or “HVR,” and are known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” as used herein refers to an immunogenic molecule that provokes an immune response. This immune response may involve antibody production, activation of specific immunologically-competent cells, or both. An antigen may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” includes any molecule, structure, amino acid sequence or protein determinant that is recognized and specifically bound by a cognate binding molecule, such as a chimeric antigen receptor, or other binding molecule, domain or protein.

A “binding domain” (also referred to as a “binding region”), as used herein, refers to a molecule or portion thereof that possesses the ability to specifically and non-covalently associate, unite, or combine with a target, such as an scFv. A binding domain includes any naturally occurring, synthetic, semi-synthetic, or recombinantly produced binding partner for a biological molecule, a molecular complex, or other target of interest. Exemplary binding domains include single chain immunoglobulin variable regions, receptor ectodomains, ligands, or synthetic polypeptides selected for their specific ability to bind to a biological molecule, a molecular complex or other target of interest.

As used herein, an “effector domain” is an intracellular portion or domain of a CAR or receptor that can directly or indirectly promote a biological or physiological response in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

A “transmembrane region,” as used herein, is a portion of a transmembrane protein that can insert into or span a cell membrane.

“Treat” or “treatment” or “ameliorate” refers to medical management of a disease, disorder, or condition of a subject. In general, an appropriate dose or treatment regimen comprising a host cell expressing a CAR of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof.

As used herein, “hyperproliferative disorder” and “proliferative disorder” refer to excessive growth or proliferation as compared to a normal or undiseased cell. Exemplary hyperproliferative disorders and proliferative disorders include tumors, cancers, neoplastic tissue, carcinoma, sarcoma, malignant cells, pre malignant cells.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

A “therapeutically effective amount” or “effective amount” of a host cell expressing a CAR of this disclosure, refers to an amount of CAR expressing host cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

The term “pharmaceutically acceptable excipient or carrier” or “physiologically acceptable excipient or carrier” refer to biologically compatible vehicles, which are described in greater detail herein, that are suitable for administration to a human or other non-human mammalian subject and generally recognized as safe or not causing a serious adverse event.

As used herein, the term “adoptive immune therapy” or “adoptive immunotherapy” refers to administration of naturally occurring or genetically engineered, disease-antigen-specific immune cells, such as T cells. Adoptive cellular immunotherapy may be autologous (immune cells are from the recipient), allogeneic (immune cells are from a donor of the same species) or syngeneic (immune cells are from a donor genetically identical to the recipient).

A “T cell” or “T lymphocyte” is an immune system cell that matures in the thymus and produces T cell receptors (TCRs), including αβT cells and γδT cells. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or T_{CM}).

A natural killer cell (“NK cells”) as used herein refers to cells which are activated in response to interferons or macrophage-derived cytokines, contain viral infections while the adaptive immune response is generating antigen-specific cytotoxic T cells that can clear the infection, and express CD56.

In addition, it should be understood that the individual constructs, or groups of constructs, derived from the various combinations of the structures and subunits described herein, are disclosed by the present disclosure to the same extent as if each construct or group of constructs was set forth individually. Thus, selection of particular structures or particular subunits is within the scope of the present disclosure.

The terminology used in the description is intended to be interpreted in its broadest reasonable manner, even though it is being used in conjunction with a detailed description of identified embodiments.

### Chimeric Antigen Receptors (CARs)

Aspects of the present disclosure are directed to chimeric antigen receptors (CAR) which bind to at least a portion of B7-H3 that is expressed at least partially on an extracellular surface of a cell, such as a malignant cell.

In certain aspects, the present disclosure provides CARs comprising (a) an extracellular region comprising a binding domain that specifically binds to at least a portion of B7-H3, (b) a transmembrane region, and (c) an intracellular region comprising an effector domain or a portion or variant thereof and a costimulatory domain or a portion or variant thereof.

B7-H3 binding domains disposed in the (a) extracellular regions of the present disclosure are, in some embodiments, scFvs which comprise at least a portion of an antibody V_{L} chain, at least a portion of an antibody V_{H} chain, and a linker domain. In some embodiments, the at least a portion of the antibody V_{L} chain is a V_{L} domain and the at least a portion of the V_{H} chain is the V_{H} domain. In some embodiments, the linker domain is a peptide linker disposed between the V_{L} domain and the V_{H} domain. For example, the B7-H3 scFvs may be designed so that the C-terminal end of the V_{L} domain is linked to the N-terminal end of the V_{H} domain by the peptide linker ((N)V_{L}(C)-linker-(N)V_{H}(C)) or such that the C-terminal end of the V_{H} domain is linked to the N-terminal end of the V_{L} domain by the peptide linker (N)V_{H}(C)-linker-(N)V_{L}(C). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser). Linker length may be varied to maximize B7-H3 antigen recognition based on B7-H3, the selected binding epitope of B7-H3, or B7-H3 antigen binding domain size and affinity (Guest et al., 2005; Patel et al. 1999; PCT Publ. No. WO 2014/031687.

Sources of binding domains specific for B7-H3 are known in the art, including known antibodies, methods of generating B7-H3 antibodies, and B7-H3 binding domains described herein. Exemplary binding domains specific for B7-H3 antigens, including CDRs thereof, are disclosed at SEQ ID NOs: 7-18. In certain embodiments, the B7-H3 scFV or variant thereof comprises or consists of an amino acid sequence having at least 75%, 80%, 85%, 90%, 91%, 92%, 93%, 94%, 95%, 96%, 97%, 98%, 99%, or more identity to any one of SEQ ID NOs: 7-18.

B7-H3 CARs of the present disclosure comprise one or more CDRs, such as three heavy chain CDRs and three light chain CDRs, according to any one of these exemplary binding domain SEQ ID NOs: 7-18, or can comprise a portion or a variant sequence thereof. B7-H3 binding domain affinities can be determined using a variety of known assays, such as Western blot, ELISA, analytical ultracentrifugation, spectroscopy and surface plasmon resonance (Biacore®) analysis (see, e.g., Scatchard et al. 1949; Wilson 2002; Wolff et al., 1993; and U.S. Pat. Nos. 5,283,173, 5,468,614, or the equivalent).

In some embodiments, the (a) extracellular region further comprises a leader domain, which includes but is not limited to, a leader peptide. For example, the leader peptide can be an IgG1 V_{K} leader domain, or a portion or variant thereof bound to the N-terminal end of the V_{H} domain or the V_{L} domain of the B7-H3 scFv.

In some embodiments, the (c) intracellular region effector domain is from CD3ζ or a functional portion or variant thereof. An exemplary CD3ζ effector domain is disclosed at SEQ ID NO: 4. In certain embodiments, the CD3ζ effector domain portion or variant thereof comprises or consists of an amino acid sequence having at least 75%, 80%, 85%, 90%, 91%, 92%, 93%, 94%, 95%, 96%, 97%, 98%, 99%, or more identity to SEQ ID NO: 4.

In some embodiments, the (c) intracellular region costimulatory domain is an intracellular tail from CD28 or portion or variant thereof and/or from an intracellular tail from 4-1 BB or portion or variant thereof. The costimulatory domain is disposed between the CD8α transmembrane domain or portion or variant thereof and the CD3ζ effector domain portion or variant thereof. When the costimulatory domain comprises both the intracellular tail from CD28 or portion or variant thereof and the intracellular tail from 4-1 BB or portion or variant thereof, the CD28 intracellular tail or portion or variant thereof can be N-terminal or C-terminal to the 4-1BB intracellular tail or portion or variant thereof. In certain embodiments, the intracellular tail from CD28 or portion or variant thereof comprises or consists of an amino acid sequence having at least 75%, 80%, 85%, 90%, 91%, 92%, 93%, 94%, 95%, 96%, 97%, 98%, 99%, or more identity to SEQ ID NO: 2. In certain embodiments, the intracellular tail from 4-1 BB or portion or variant thereof comprises or consists of an amino acid sequence having at least 75%, 80%, 85%, 90%, 91%, 92%, 93%, 94%, 95%, 96%, 97%, 98%, 99%, or more identity to SEQ ID NO: 3.

The (a) extracellular region and the (c) intracellular region of the present disclosure are connected by the (b) transmembrane region. For example, the (b) a transmembrane region is disposed between a C-terminal end of the B7-H3 scFv and an N-terminal end of the (c) intracellular region. In certain embodiments, the transmembrane region comprises or is derived from a known transmembrane protein, such as a CD8α transmembrane region. In some embodiments, the (b) transmembrane region comprises a known hinge domain and a known transmembrane domain, such as the hinge domain and the transmembrane domain of CD8α, or a portion or variant thereof. An exemplary CD8α hinge domain and CD8α transmembrane domain are disclosed at SEQ ID NO: 1. In certain embodiments, the CD8α hinge domain and CD8α transmembrane domain portion or variant thereof comprises or consists of an amino acid sequence having at least 75%, 80%, 85%, 90%, 91%, 92%, 93%, 94%, 95%, 96%, 97%, 98%, 99%, or more identity to SEQ ID NO: 1.

Polypeptide markers are unique peptide sequences that are co-expressed in a cell, such as a host cell, along with one or more B7-H3 CARs. Unlike B7-H3 CARs of the present disclosure, polypeptide markers are recognized or bound by, for example, an antibody or detected by an emitted signal, such as fluorescence. Polypeptide markers can be useful for detecting, identifying, isolating, tracking, purifying, enriching for, targeting, or biologically or chemically modifying tagged proteins of interest, particularly when a tagged protein is part of a heterogeneous population of cell proteins or cells, such as a biological sample like peripheral blood. Exemplary polypeptide markers of the present disclosure include green fluorescent protein (GFP) and a truncated form of epidermal growth factor receptor (EGFRt). Amino acid sequences of GFP are generally known in the art and an exemplary EGFRt amino acid sequence is shown as SEQ ID NO: 6.

Table 1 below lists amino acid sequences of various 1B7-H-3 CAR components. For each mAb sequence, the leader sequence is shown italicized, the FR1-4 sequences are shown underlined, and the CDR1-3 sequences are shown in bold.

TABLE 1
B7-H3 CAR Amino Acid Sequences

| SEQ | | |
|---|---|---|
| ID NO. | NAME | SEQUENCE |
| 1 | CD8α hinge and | TTTPAPRPPTPAPTIASQPLSLRPEACRPAAGGAVHTRGL |
| | CD8α | DFACDIYIWAPLAGTCGVLLLSLVITLYC |
| | transmembrane | |
| | region | |
| 2 | CD28 | RSKRSRLLHSDYMNMTPRRPGPTRKHYQPYAPPRDFAAY |
| | intracellular tail | RS |
| 3 | 4-1BB | KRGRKKLLYIFKQPFMRPVQTTQEEDGCSCRFPEEEEGG |
| | intracellular tail | CEL |
| 4 | CD3ζ | RVKFSRSADAPAYQQGQNQLYNELNLGRREEYDVLDKRR |
| | | GRDPEMGGKPQRRKNPQEGLYNELQKDKMAEAYSEIGM |
| | | KGERRRGKGHDGLYQGLSTATKDTYDALHMQALPPR |
| 5 | P2A | GSGATNFSLLKQAGDVEENPGP |
| 6 | EGFRt | RKVCNGIGIGEFKDSLSINATNIKHFKNCTSISGDLHILPVAF |
| | | RGDSFTHTPPLDPQELDILKTVKEITGFLLIQAWPENRTDL |
| | | HAFENLEIIRGRTKQHGQFSLAVVSLNITSLGLRSLKEISDG |
| | | DVIISGNKNLCYANTINWKKLFGTSGQKTKIISNRGENSCK |
| | | ATGQVCHALCSPEGCWGPEPRDCVSCRNVSRGRECVDK |
| | | CNLLEGEPREFVENSECIQCHPECLPQAMNITCTGRGPDN |
| | | CIQCAHYIDGPHCVKTCPAGVMGENNTLVWKYADAGHVC |
| | | HLCHPNCTYGCTGPGLEGCPTNGPKIPSIATGMVGALLLL |
| | | LVVALGIGLFM |
| 7 | 1G5 mAb | MGWSYIILFLVATATGVHSQVQLQQPGAELVKPGASVKLS |
| | (V_{H} chain; | CKASGYTFTSYWMHWVKQRPGQGLEWIGMIHPNSGSTN |
| | leader | YNEKFKSKATLTVDKSSSTAYMQLSSLTSEDSAVYYCARG |
| | sequence, | GYFDYWGQGTTLTVSS |
| | CDRs 1-3, and | |
| | FRs 1-4) | |
| 8 | 1G5 mAb | MKTQTQVFVYMLLWLSGVDGDIVMTQSQKFMSTSVGDRV |
| | (V_{L} chain; leader | SVTCKASQNVGTNVAWYQQKPGQSPKALIYSASYRYSGV |
| | sequence, | PDRFTGSGSGTDFTLTISNVQSEDLAEYFCQQYNSYPYTF |
| | CDRs 1-3, and | GGGTKLEIKR |
| | FRs 1-4) | |
| 9 | 23B2 mAb | MGWSCIMLFLVATATGVHSQVQLQQPGAELVKPGASVKL |
| | (V_{H} chain; | SCKASGYTFTSYWMHWVKQRPGQGLEWIGMIHPNSGST |
| | leader | NYNEKFKSKATLTVDKSSSTAYMQLSSLTSEDSAVYYCAR |
| | sequence, | ESRQLDYWGQGTTLTVSS |
| | CDRs 1-3, and | |
| | FRs 1-4) | |
| 10 | 23B2 mAb | MDLQVQIFSFLLISASVILSRGQIVLTQSPAIMSASPGEKVT |
| | (V_{L} chain; leader | MTCSASSSISYMHWYQQKPGTSPKRWIYDTSKLASGVPA |
| | sequence, | RFSGSGSGTSYSLTISSMEAEDAATYYCHQRSSYPLTFGA |
| | CDRs 1-3, and | GTKLELK |
| | FRs 1-4) | |
| 11 | 15F9 mAb | MGWSCIMFFLVATATGVHSQVQLQQPGAELVKPGASVKL |
| | (V_{H} chain; | SCKASGYTFTNYWMHWVKQRPGQGLEWIGMIHPNSGST |
| | leader | NYNEKFKSKATLTVDKSSSTAYMQLSSLTSEDSAVYYCAR |
| | sequence, | RGDGDYWGQGTTLTVSS |
| | CDRs 1-3, and | |
| | FRs 1-4) | |
| 12 | 15F9 mAb | MVLISLLFWFPGTRCDIQMTRHPSSLSASMGERVSLTCRA |
| | (V_{L} chain; leader | SQEISGYLSWLQQKPDGTIKRLIYAASTLDSGVPKRFSGS |
| | sequence, | RSGSDYSLTISSLESEDFADYYCLQYASYPYTFGGGTKLEI |
| | CDRs 1-3, and | |
| | FRs 1-4) | |
| 13 | 8B12 mAb | MEWSWVILILVAAATGVHSQVQLQQPGAELVKPGASVKM |
| | (V_{H} chain; | SCKASGYTFTSYWITWVKQRPGQGLEWIGDIYPGSGSTN |
| | leader | YNEKFKSKATLTVDTSSSTAYMQLSSLTSEDSAVYYCARG |
| | sequence, | GTRFSPFAYWGQGTLVTVSAA |
| | CDRs 1-3, and | |
| | FRs 1-4) | |
| 14 | 8B12 mAb | MKMPVRLLVLMFWIPASSSDVLMTQTPLSLPVSLGDQASI |
| | (V_{L} chain; leader | SCRSSQSIVHSNGNTYLEWYLQKPGQSPKLLIYKVSNRFS |
| | sequence, | GVPDRFSGSGSGTDFTLKISRVEAEDLGVYYCFQGSHVP |
| | CDRs 1-3, and | WTFGGGTKLEIKRADA |
| | FRs 1-4) | |
| 15 | 12B4 mAb | MGWSCIMFFLVATATGVHSQVQLQQPGAELVKPGASVKL |
| | (V_{H} chain; | SCKASGYTFTSYWMHWVKQRPGQGLEWIGMIHPNSGST |
| | leader | NYNEKFKSKATLTVDKSSSTAYMQLSSLTSEDSAVYYCAR |
| | sequence, | YYYGSSYAMDYWGQGTSVTVS |
| | CDRs 1-3, and | |
| | FRs 1-4) | |
| 16 | 12B4 mAb | MDFQVQIFSFLLISASVIMSRGQIVLTQSPAIMSASPGEKVT |
| | (V_{L} chain; leader | ITCSASSSVSYMHWFQQKPGTSPKLWIYSTSNLASGVPA |
| | sequence, | RFSGSGSGTSYSLTISRMEAEDAATYYCQQRSSYPYTFG |
| | CDRs 1-3, and | GGTKLEIKRAD |
| | FRs 1-4) | |
| 17 | 24D12 mAb | MGWSCIMFFLVATATGVHSQVQLQQPGAELVKPGASVKL |
| | (V_{H} chain; | SCKASGYTFTSYWMHWVKQRPGQGLEWIGMIHPNSGST |
| | leader | NYNEKFKSKATLTVDKSSSTAYMQLSSLTSEDSAVYYCAN |
| | sequence, | YYGTNVWGTGTTVTVS |
| | CDRs 1-3, and | |
| | FRs 1-4) | |
| 18 | 24D12 mAb | METDTLLLWVLLLWVPGSTGDIVLTQSPASLAVSLGQRATI |
| | (V_{L} chain; leader | SYRASKSVSTSGYSYMHWNQQKPGQPPRLLIYLVSNLES |
| | sequence, | GVPARFSGSGSGTDFTLNIHPVEEEDAATYYCQHIREAYT |
| | CDRs 1-3, and | FGRGDQSWK |
| | FRs 1-4) | |

### Polynucleotides, Vectors, and Host Cells

Nucleic acid molecules and polynucleotides are provided that encode any one or more of the 1B7-H-3 CARs or variants or portions thereof as described herein. In certain embodiments, a polynucleotide encoding at least a portion of the 1B7-H-3 CARs of the present disclosure comprises, or consists of, a polynucleotide having at least 70%, 75%, 80%, 85%, 90%, 91%, 92%, 93%, 94%, 95%, 96%, 97%, 98%, 99%, or more identity to the nucleotide sequence set forth in any one of SEQ ID NOs: 19-22 and 25-36.

In certain embodiments, a polynucleotide encoding a B7-H3 CAR comprises a polynucleotide that encodes a CD3ζ domain that has at least 70%, 75%, 80%, 85%, 90%, 91%, 92%, 93%, 94%, 95%, 96%, 97%, 98%, 99%, or more identity to SEQ ID NO: 22.

In certain embodiments, a polynucleotide encoding a B7-H3 CAR comprises a polynucleotide that encodes a 4-1 BB intracellular tail that has at least 70%, 75%, 80%, 85%, 90%, 91%, 92%, 93%, 94%, 95%, 96%, 97%, 98%, 99%, or more identity to SEQ ID NO: 21.

In certain embodiments, a polynucleotide encoding a B7-H3 CAR comprises a polynucleotide that encodes a CD28 intracellular tail that has at least 70%, 75%, 80%, 85%, 90%, 91%, 92%, 93%, 94%, 95%, 96%, 97%, 98%, 99%, or more identity to SEQ ID NO: 20.

In certain embodiments, a polynucleotide encoding a B7-H3 CAR comprises a polynucleotide that encodes a CD8α hinge and CD8α transmembrane region that has at least 70%, 75%, 80%, 85%, 90%, 91%, 92%, 93%, 94%, 95%, 96%, 97%, 98%, 99%, or more identity to SEQ ID NO: 19.

In certain embodiments, a polynucleotide encoding a B7-H3 CAR comprises a polynucleotide that encodes at least a portion of a B7-H3 binding domain that has at least 70%, 75%, 80%, 85%, 90%, 91%, 92%, 93%, 94%, 95%, 96%, 97%, 98%, 99%, or more identity to any one or more of SEQ ID NOs: 25-36, such as, but not limited to, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 35 and SEQ ID NO: 36.

Polynucleotides of the present disclosure further include polynucleotides encoding a marker, such as GFP and/or EGFRt. In certain embodiments, a polynucleotide encoding EGFRt comprises a polynucleotide that has at least 70%, 75%, 80%, 85%, 90%, 91%, 92%, 93%, 94%, 95%, 96%, 97%, 98%, 99%, or more identity to SEQ ID NO: 24.

In some embodiments, polynucleotides of the present disclosure further include polynucleotides encoding a self-cleaving peptide, such as a 2A peptide. An exemplary 2A peptide is P2A encoded by a polynucleotide that has at least 70%, 75%, 80%, 85%, 90%, 91%, 92%, 93%, 94%, 95%, 96%, 97%, 98%, 99%, or more identity to SEQ ID NO: 23.

Table 2 below lists the nucleotide sequences of various B7-H3 CAR components. For each mAb sequence, the leader sequence is shown italicized, the FR1-4 sequences are shown underlined, and the CDR1-3 sequences are shown in bold.

TABLE 2
B7-H3 CAR Nucleotide Sequences

| SEQ | | |
|---|---|---|
| ID NO. | NAME | SEQUENCE |
| 19 | CD8α hinge and | accacgacgccagcgccgcgaccaccaacaccggcgcccaccatcgcgtcgc |
| | CD8α | agcccctgtccctgcgcccagaggcgtgccggccagcggcggggggcgcagt |
| | transmembrane | gcacacgagggggctggacttcgcctgtgatatctacatctgggcgcccttggccg |
| | region | ggacttgtggggtccttctcctgtcactggttatcaccctttactgc |
| 20 | CD28 | aggagtaagaggagcaggctcctgcacagtgactacatgaacatgactccccg |
| | intracellular tail | ccgccccgggcccacccgcaagcattaccagccctatgccccaccacgcgactt |
| | | cgcagcctatcgctcc |
| 21 | 4-1BB | aaacggggcagaaagaaactcctgtatatattcaaacaaccatttatgagacca |
| | intracellular tail | gtacaaactactcaagaggaagatggctgtagctgccgatttccagaagaagaa |
| | | gaaggaggatgtgaactg |
| 22 | CD3ζ | agagtgaagttcagcaggagcgcagacgcccccgcgtaccagcagggccag |
| | | aaccagctctataacgagctcaatctaggacgaagagaggagtacgatgttttgg |
| | | acaagagacgtggccgggaccctgagatggggggaaagccgcagagaagg |
| | | aagaaccctcaggaaggcctgtacaatgaactgcagaaagataagatggcgg |
| | | aggcctacagtgagattgggatgaaaggcgagcgccggaggggcaaggggc |
| | | acgatggcctttaccagggtctcagtacagccaccaaggacacctacgacgccc |
| | | ttcacatgcaggccctgccccctcgctaa |
| 23 | P2A | ggaagcggagctactaacttcagcctgctgaagcaggctggagacgtggagga |
| | | gaaccctggacct |
| 24 | EGFRt | cgcaaagtgtgtaacggaataggtattggtgaatttaaagactcactctccataaat |
| | | gctacgaatattaaacacttcaaaaactgcacctccatcagtggcgatctccacat |
| | | cctgccggtggcatttaggggtgactccttcacacatactcctcctctggatccaca |
| | | ggaactggatattctgaaaaccgtaaaggaaatcacagggtttttgctgattcagg |
| | | cttggcctgaaaacaggacggacctccatgcctttgagaacctagaaatcatacg |
| | | cggcaggaccaagcaacatggtcagttttctcttgcagtcgtcagcctgaacataa |
| | | catccttgggattacgctccctcaaggagataagtgatggagatgtgataatttcag |
| | | gaaacaaaaatttgtgctatgcaaatacaataaactggaaaaaactgtttgggac |
| | | ctccggtcagaaaaccaaaattataagcaacagaggtgaaaacagctgcaag |
| | | gccacaggccaggtctgccatgccttgtgctcccccgagggctgctggggcccg |
| | | gagcccagggactgcgtctcttgccggaatgtcagccgaggcagggaatgcgtg |
| | | gacaagtgcaaccttctggagggtgagccaagggagtttgtggagaactctgagt |
| | | gcatacagtgccacccagagtgcctgcctcaggccatgaacatcacctgcacag |
| | | gacggggaccagacaactgtatccagtgtgcccactacattgacggcccccact |
| | | gcgtcaagacctgcccggcaggagtcatgggagaaaacaacaccctggtctgg |
| | | aagtacgcagacgccggccatgtgtgccacctgtgccatccaaactgcacctac |
| | | ggatgcactgggccaggtcttgaaggctgtccaacgaatgggcctaagatcccgt |
| | | ccatcgccactgggatggtgggggccctcctcttgctgctggtggtggccctgggg |
| | | atcggcctcttcatg |
| 25 | 1G5 mAb | atgggatggagctatatcatcctctttttggtagcaacagctacaggtgtccactccc |
| | (V_{H} chain; | aggtccaactgcagcagcctggggctgagctggtaaagcctggggcttcagtga |
| | leader | agttgtcctgcaaggcttctggctacactttcaccagctactggatgcactgggt |
| | sequence, | gaagcagaggcctggacaaggccttgagtggattggaatgattcatcctaatag |
| | CDRs 1-3, and | tggtagtactaactacaatgagaagttcaagagcaaggccacactgactgta |
| | FRs 1-4) | gacaaatcctccagcacagcctacatgcaactcagcagcctgacatctgaggac |
| | | tctgcggtctattactgtgcaaggggggggtactttgactactggggccaaggc |
| | | accactctcacagtctcctca |
| 26 | 1G5 mAb | atgaagacacagactcaggtctttgtatacatgttgctgtggttgtctggtgttgatgg |
| | (V_{L} chain; leader | agacattgtgatgacccagtctcaaaaattcatgtccacatcagtaggagacagg |
| | sequence, | gtcagcgtcacctgcaaggccagtcagaatgtgggtactaatgtagcctggta |
| | CDRs 1-3, and | tcaacagaaaccagggcaatctcctaaagcactgatttactcggcatcctaccg |
| | FRs 1-4) | gtacagtggagtccctgatcgcttcacaggcagtggatctgggacagatttcactc |
| | | tcaccatcagcaatgtgcagtctgaagacttggcagagtatttctgtcagcaatata |
| | | acagctatccgtacacgttcggaggggggaccaagctggaaataaaacgg |
| 27 | 23B2 mAb | atgggatggagctgtatcatgctctttttggtagcaacagctacaggtgtccactccc |
| | (V_{H} chain; | aggtccaactgcagcagcctggggctgagctggtaaagcctggggcttcagtga |
| | leader | agttgtcctgcaaggcttctggctacactttcaccagctactggatgcactgggt |
| | sequence, | gaagcagaggcctggacaaggccttgagtggattggaatgattcatcctaatag |
| | CDRs 1-3, and | tggtagtactaactacaatgagaagttcaagagcaaggccacactgactgta |
| | FRs 1-4) | gacaaatcctccagcacagcctacatgcaactcagcagcctgacatctgaggac |
| | | tctgcggtctattactgtgcaagagagagtagacagctcgactactggggcca |
| | | aggcaccactctcacagtctcctca |
| 28 | 23B2 mAb | atggatttacaagtgcagattttcagcttcctgctaatcagtgcctcagtcatactgtc |
| | (V_{L} chain; leader | cagaggacaaattgttctcacccagtctccagcaatcatgtctgcatctccaggg |
| | sequence, | gagaaggtcaccatgacctgcagtgccagctcaagtataagttacatgcac |
| | CDRs 1-3, and | tggtaccagcagaagccaggcacctcccccaaaagatggatttatgacacatc |
| | FRs 1-4) | caaactggcttctggagtccctgctcgcttcagtggcagtgggtctgggacctctta |
| | | ttctctcacaatcagcagcatggaggctgaagatgctgccacttattactgccatca |
| | | gcggagtagttacccgctcacgttcggtgctgggaccaagctggagctgaaa |
| 29 | 15F9 mAb | atgggatggagctgtatcatgttctttttggtagcaacagctacaggtgtccactccc |
| | (V_{H} chain; | aggtccaactgcagcagcctggggctgagctggtaaagcctggggcttcagtga |
| | leader | agttgtcctgcaaggcttctggctacactttcaccaactactggatgcactgggt |
| | sequence, | gaagcagaggcctggacaaggccttgagtggattggaatgattcatcctaatag |
| | CDRs 1-3, and | tggtagtactaactacaatgagaagttcaagagcaaggccacactgactgta |
| | FRs 1-4) | gacaaatcctccagcacagcctacatgcaactcagcagcctgacatctgaggac |
| | | tctgcggtctattactgtgcaaggcgaggggacggggactactggggccaagg |
| | | caccactctcacagtctcctcag |
| 30 | 15F9 mAb | atggttcttatttccttgctgttctggtttccaggtaccagatgtgacatccagatgacc |
| | (V_{L} chain; leader | cggcatccttcttctttatctgcctctatgggagaaagagtcagtctcacttgtcgggc |
| | sequence, | aagtcaggaaattagtggttacttaagctggcttcagcagaaaccagatggaa |
| | CDRs 1-3, and | ctattaaacgcctgatttacgccgcatccactttagattctggtgtcccaaaaagg |
| | FRs 1-4) | ttcagtggcagtaggtctgggtcagattattctctcaccatcagcagccttgagtctg |
| | | aagattttgcagactattactgtctacaatatgctagttatccgtacacgttcggag |
| | | gggggaccaagctggaaata |
| 31 | 8B12 mAb | atggaatggagctgggtcatcctcattttggtagcagcagctacaggtgtccactcc |
| | (V_{H} chain; | caggtccaactgcagcagcctggggctgagcttgtgaagcctggggcttcagtga |
| | leader | agatgtcctgcaaggcttctggctacaccttcaccagctactggataacctggg |
| | sequence, | tgaagcagaggcctggacaaggccttgagtggattggagatatttatcctggta |
| | CDRs 1-3, and | gtggtagtactaactacaatgagaagttcaagagcaaggccactctgactgta |
| | FRs 1-4) | gacacatcctccagcacagcctacatgcagctcagcagcctgacatctgaggac |
| | | tctgcggtctattactgtgcaagagggggtactagattcagcccctttgcttact |
| | | ggggccaagggactctggtcactgtctctgcagctac |
| 32 | 8B12 mAb | atgaagatgcctgttaggctgttggtgctgatgttctggattcctgcttccagcagtga |
| | (V_{L} chain; leader | tgttttgatgacccaaactccactctccctgcctgtcagtcttggagatcaagcctcc |
| | sequence, | atctcttgcagatctagtcagagcattgtacatagtaatggaaacacctatttag |
| | CDRs 1-3, and | aatggtacctgcagaaaccaggccagtctccaaagctcctgatctacaaagtttc |
| | FRs 1-4) | caaccgattttctggggtcccagacaggttcagtggcagtggatcagggacaga |
| | | tttcacactcaagatcagcagagtggaggctgaggatctgggagtttattactgcttt |
| | | caaggttcacatgttccgtggacgttcggtggaggcaccaagctggaaatcaa |
| | | acgggctgatgct |
| 33 | 12B4 mAb | atgggatggagctgtatcatgttctttttggtagcaacagctacaggtgtccactccc |
| | (V_{H} chain; | aggtccaactgcagcagcctggggctgagctggtaaagcctggggcttcagtga |
| | leader | agttgtcctgcaaggcttctggctacactttcaccagctactggatgcactgggt |
| | sequence, | gaagcagaggcctggacaaggccttgagtggattggaatgattcatcctaatag |
| | CDRs 1-3, and | tggtagtactaactacaatgagaagttcaagagcaaggccacactgactgta |
| | FRs 1-4) | gacaaatcctccagcacagcctacatgcaactcagcagcctgacatctgaggac |
| | | tctgcggtctattactgtgcaagatattactacggtagtagctatgctatggacta |
| | | ctggggtcaaggaacctcagtcaccgtctctca |
| 34 | 12B4 mAb | atggattttcaggtgcagattttcagcttcctgctaatcagtgcctcagtcataatgtcc |
| | (V_{L} chain; leader | agaggacaaattgttctcacccagtctccagcaatcatgtctgcatctccagggga |
| | sequence, | gaaggtcaccataacctgcagtgccagctcaagtgtaagttacatgcactggtt |
| | CDRs 1-3, and | ccagcagaagccaggcacttctcccaaactctggatttatagcacatccaacct |
| | FRs 1-4) | ggcttctggagtccctgctcgcttcagtggcagtggatctgggacctcttactctctc |
| | | acaatcagccgaatggaggctgaagatgctgccacttattactgccagcaaagg |
| | | agtagttacccgtacacgttcggaggggggaccaagctggaaataaaacggg |
| | | ctgatg |
| 35 | 24D12 mAb | atgggatggagctgtatcatgttctttttggtagcaacagctacaggtgtccactccc |
| | (V_{H} chain; | aggtccaactgcagcagcctggggctgagctggtaaagcctggggcttcagtga |
| | leader | agttgtcctgcaaggcttctggctacactttcaccagctactggatgcactgggt |
| | sequence, | gaagcagaggcctggacaaggccttgagtggattggaatgattcatcctaatag |
| | CDRs 1-3, and | tggtagtactaactacaatgagaagttcaagagcaaggccacactgactgta |
| | FRs 1-4) | gacaaatcctccagcacagcctacatgcaactcagcagcctgacatctgaggac |
| | | tctgcggtctattactgtgcaaattactacggtactaatgtctggggcacagggac |
| | | cacggtcaccgtctct |
| 36 | 24D12 mAb | atggagacagacacactgctgctatgggtactgctgctctgggttccaggttccact |
| | (V_{L} chain; leader | ggtgacattgtgctgacacagtctcctgcttccttagctgtatctctggggcagaggg |
| | sequence, | ccaccatctcatacagggccagcaaaagtgtcagtacatctggctatagttata |
| | CDRs 1-3, and | tgcactggaaccaacagaaaccaggacagccacccagactcctcatctatcttg |
| | FRs 1-4) | tatccaacctagaatctggggtccctgccaggttcagtggcagtgggtctgggac |
| | | agacttcaccctcaacatccatcctgtggaggaggaggatgctgcaacctattact |
| | | gtcagcacattagggaagcttacacgttcggaaggggggaccaaagctgga |
| | | aa |

In any of the embodiments described herein, B7-H3 CARs, portions, or variants thereof may be codon-optimized for a host cell containing the polynucleotide using known techniques (Scholten et al., 2006). Codon optimization can be performed using, e.g., the GenScript® OptimumGene™ tool. Codon-optimized sequences include sequences that are partially or fully codon-optimized.

A polynucleotide encoding a B7-H3 CAR of this disclosure can be inserted into an expression vector, such as a viral vector, for transduction into a host cell, such as a T cell. In some embodiments, an expression construct of the present disclosure comprises a B7-H3 CAR polynucleotide (e.g., SEQ ID NOs: 19-22 plus one or more of SEQ ID NOs:25-36 or portions there) and optionally a P2A self-cleaving peptide (e.g., SEQ ID NO. 23) and optionally an EGFRt marker (e.g., SEQ ID No. 24) operably linked to an expression control sequence such as a promoter.

In certain embodiments, polynucleotides of the present disclosure may be operatively linked to certain elements of the vector. For example, polynucleotide sequences that are needed to affect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the expression construct is comprised in a vector which may integrate into a host cell's genome or promote integration of the polynucleotide insert upon introduction into the host cell and thereby replicate along with the host genome, such as a viral vector. Viral vectors include retrovirus, adenovirus, parvovirus, coronavirus, negative strand RNA viruses, positive strand RNA viruses, and double-stranded DNA viruses. (Coffin, J. M., Retroviridae: The viruses and their replication, In Fundamental Virology, Third Edition, B. N. Fields et al., Eds., Lippincott-Raven Publishers, Philadelphia, 1996).

Construction of an expression vector that is used for genetically engineering and producing a CAR of interest can be accomplished by using any suitable molecular biology engineering techniques known in the art. To obtain efficient transcription and translation, a polynucleotide in each recombinant expression construct includes at least one appropriate expression control sequence, such as a leader sequence and particularly a promoter operably linked to the nucleotide sequence encoding the immunogen. Methods for making CARs of the present disclosure are described, for example, in U.S. Pat. Nos. 6,410,319; 7,446,191; U.S. Patent Publ. No. 2010/065818; U.S. Pat. No. 8,822,647; PCT Publ. No. WO 2014/031687; U.S. Pat. No. 7,514,537; Brentjens et al., 2007; and Walseng et al., 2017; the techniques of which are herein incorporated by reference.

In certain embodiments, polynucleotides of the present disclosure are used to transfect/transduce a host cell, such as a T cell or an NK cell, for use in adoptive transfer therapy to target B7-H3. Cells may be induced to incorporate the vector or other material by use of a viral vector, transformation via calcium phosphate precipitation, DEAE-dextran, electroporation, microinjection, or other methods. (Sambrook et al., Molecular Cloning: A Laboratory Manual 2d ed. (Cold Spring Harbor Laboratory, 1989)). T cells and/or NK cells can be collected using known techniques, and the various subpopulations or combinations thereof can be enriched or depleted by known techniques, such as by affinity binding to antibodies, flow cytometry, or immunomagnetic selection. In certain embodiments, the T cell is a CD4+ T cell, a CD8+ T cell, a CD4− CD8− double negative T cell, a naïve T cell, a central memory T cell, an effector memory T cell, a stem cell memory T cell, or any combination thereof. Methods for transfecting/transducing T cells with polynucleotides have been previously described (U.S. Patent Application Pub. No. US 2004/0087025) as have adoptive transfer procedures using T cells of desired target-specificity (Schmitt et al. 2009; Dossett et al. 2009; Till et al. 2008; Wang et al. 2007; Kuball et al., 2007; Leen et al., 2007; U.S. Patent Publ. No. 2011/0243972; U.S. Patent Publ. No. 2011/0189141), such that adaptation of these methodologies to the presently disclosed B7-H3 CARs of the present disclosure is within the scope of the present disclosure.

In certain embodiments, a B7-H3 CAR of the instant disclosure is expressed by a host cell, such as a T cell and/or a NK cell, and the host cell recognizes and initiates an immune response to a target cell expressing B7-H3. As explained in greater detail below, the target cell includes malignant cells, such as cancer cells.

In any of the embodiments disclosed herein, B7-H3 CARs, when expressed by a host cell such as a T cell and/or a NK cell, results in at least one of the following outcomes (i) improved cell signaling, cytotoxic activity, proliferation, and/or survival in response to B7-H3 relative to a T cell and/or a NK cell that does not express the B7-H3 CAR of the present disclosure, wherein improved cell signaling optionally comprises increased and/or sustained cytokine production and/or release, and/or phosphorylation of one or more protein associated with an immune cell response to antigen-binding, or any combination thereof; (ii) improved cell activity in response to antigen relative to a T cell and/or a NK cell that does not express the B7-H3 CAR of the present disclosure, wherein improved cell signaling optionally comprises increased mobilization of intracellular calcium, killing activity, proliferation, earlier activation in response to antigen, or any combination thereof; (iii) improved cell signaling and/or activity, relative to a T cell and/or a NK cell that does not express the B7-H3 CAR of the present disclosure, upon binding to a target antigen that is expressed at a low level or an intermediate level on a target cell surface; (iv) reducing or suppressing growth, area, volume, and/or spread of a tumor that expresses an antigen that is recognized and/or specifically bound by the B7-H3 CAR, of killing tumor cells, and/or of increasing survival of the subject to a greater degree and/or for a longer period of time as compared to a T cell and/or a NK cell that does not express the B7-H3 CAR of the present disclosure; (iv) improved sensitivity to B7-H3 antigen expression compared to a T cell that does not express the B7-H3 CAR of the present disclosure; or (v) any combination of (i)-(iv).

Functional characterization of B7-H3 CARs described herein may be performed according to any art-accepted methodologies for assaying T cell and/or NK cell activity, including determination of T cell and/or NK cell binding, activation or induction and also including determination of T cell and/or NK cell responses that are antigen-specific. Examples include determination of intracellular calcium, T cell proliferation, T cell and/or NK cell cytokine release, antigen-specific T cell and/or NK cell stimulation, MHC-restricted T cell and/or NK cell stimulation, cytotoxic activity, changes in T cell and/or NK cell phenotypic marker expression, phosphorylation of certain T cell and/or NK cell proteins, and other measures of T cell and/or NK cell functions. Procedures for performing these and similar assays are described herein and/or may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein.

In some embodiments, kits are provided comprising (a) a B7-H3 CAR vector described herein, (b) a B7-H3 CAR polynucleotide (e.g., SEQ ID NOs: 19-22 plus one or more of SEQ ID NOs:25-36 or portions there), (c) marker peptide and self-cleaving peptide (SEQ ID NOs: 23 and 24), (d) instructions, and/or (e) one or more reagents for transducing the vector or polynucleotides into a host cell.

### B7-H3 Binding Domain

In certain embodiments, the 1B7-H-3 binding domain comprises one or more heavy chain CDRs (e.g., one V_{H} CDR, two V_{H} CDRs, or three V_{H} CDRs) and/or one or more light chain CDRs (e.g., one V_{L} CDR, two V_{L} CDRs, or three V_{L} CDRs). Depending on the number of the CDRs, a corresponding number of FRs can be used. For example, if 3 CDRs are used for V_{H} or V_{L}, 4 FRs are used for the corresponding V_{H} or V_{L}, with an FR disposed at the N-terminus of the first CDR, between two CDRs, and/or at the C-terminus of the last CDR. In some embodiments, a leader having an amino acid sequence set forth by any one of SEQ ID NOs: 100-109 is added to the N-terminus of V_{H}, V_{L}, or both. In some embodiments, a linker is disposed between the V_{H} and V_{L} domains. Suitable linker sequences are disclosed in the foregoing section. One of ordinary skill in the art can select the CDR sequences from Table 3, FR sequences from Table 4, and leader sequences from Table 5 below to assemble a desirable scFv sequence.

TABLE 3
Amino Acid Sequences of CDRs
V_{H} CDR Sequences

| CDR1 | CDR2 | CDR3 |
|---|---|---|
| GYTFTSYWMH | MIHPNSGSTNYNEKFKS | GGYFDY |
| (SEQ ID NO: 37) | (SEQ ID NO: 46) | (SEQ ID NO: 54) |
| GYTFTSYWMH | MIHPNSGSTNYNEKFKS | ARESRQLDY |
| (SEQ ID NO: 37) | (SEQ ID NO: 46) | (SEQ ID NO: 55) |
| GYTFTNYWMH | MIHPNSGSTNYNEKFKS | RGDGDY |
| (SEQ ID NO: 38) | (SEQ ID NO: 46) | (SEQ ID NO: 56) |
| GYTFTSYWIT | DIYPGSGSTNYNEKFKS | ARGGTRFSPFAY |
| (SEQ ID NO: 39) | (SEQ ID NO: 47) | (SEQ ID NO: 57) |
| GYTFTSYWMH | MIHPNSGSTNYNEKFKS | YYYGSSYAMDY |
| (SEQ ID NO: 37) | (SEQ ID NO: 46) | (SEQ ID NO: 58) |
| GYTFTSYWMH | MIHPNSGSTNYNEKFKS | YYGTNV |
| (SEQ ID NO: 37) | (SEQ ID NO: 46) | (SEQ ID NO: 59) |

V_{L} CDR Sequences

| CDR1 | CDR2 | CDR3 |
|---|---|---|
| KASQNVGTNVA | SASYRYS | QQYNSYPYT |
| (SEQ ID NO: 40) | (SEQ ID NO: 48) | (SEQ ID NO: 60) |
| SASSSISYMH | DTSKLAS | HQRSSYPLT |
| (SEQ ID NO: 41) | (SEQ ID NO: 49) | (SEQ ID NO: 61) |
| RASQEISGYLS | AASTLDS | LQYASYPYT |
| (SEQ ID NO: 42) | (SEQ ID NO: 50) | (SEQ ID NO: 62) |
| RSSQSIVHSNGNTYLE | KVSNRFS | FQGSHVPWT |
| (SEQ ID NO: 43) | (SEQ ID NO: 51) | (SEQ ID NO: 63) |
| SASSSVSYMH | STSNLAS | QQRSSYPYT |
| (SEQ ID NO: 44) | (SEQ ID NO: 52) | (SEQ ID NO: 64) |
| KSVSTSGYSYMH | LVSNLES | QHIREAYT |
| (SEQ ID NO: 45) | (SEQ ID NO: 53) | (SEQ ID NO: 65) |

TABLE 4
Amino Acid Sequences of FRs
V_{H} FR Sequences

| FR1 | FR2 | FR3 | FR4 |
|---|---|---|---|
| QVQLQQPGAELVKPG | WVKQRPGQ | KATLTVDKSSSTAYMQLSSL | WGQGTTLT |
| ASVKLSCKAS | GLEWIG | TSEDSAVYYCAR | VSS |
| (SEQ ID NO: 66) | (SEQ ID NO: | (SEQ ID NO: 81) | (SEQ ID NO: |
| | 74) | | 90) |
| QVQLQQPGAELVKPG | WVKQRPGQ | KATLTVDKSSSTAYMQLSSL | WGQGTTLT |
| ASVKLSCKAS | GLEWIG | TSEDSAVYYC | VSS |
| (SEQ ID NO: 66) | (SEQ ID NO: | (SEQ ID NO: 82) | (SEQ ID NO: |
| | 74) | | 90) |
| QVQLQQPGAELVKPG | WVKQRPGQ | KATLTVDKSSSTAYMQLSSL | WGQGTTLT |
| ASVKLSCKAS | GLEWIG | TSEDSAVYYCAR | VSS |
| (SEQ ID NO: 66) | (SEQ ID NO: | (SEQ ID NO: 81) | (SEQ ID NO: |
| | 74 | | 90) |
| QVQLQQPGAELVKPG | WVKQRPGQ | KATLTVDTSSSTAYMQLSSL | WGQGTLVT |
| ASVKMSCKAS | GLEWIG | TSEDSAVYYC | VSAA |
| (SEQ ID NO: 67) | (SEQ ID NO: | (SEQ ID NO: 82) | (SEQ ID NO: |
| | 74) | | 91) |
| QVQLQQPGAELVKPG | WVKQRPGQ | KATLTVDKSSSTAYMQLSSL | WGQGTSV |
| ASVKLSCKAS | GLEWIG | TSEDSAVYYCAR | TVS |
| (SEQ ID NO: 66) | (SEQ ID NO: | (SEQ ID NO: 81) | (SEQ ID NO: |
| | 74) | | 92) |
| QVQLQQPGAELVKPG | WVKQRPGQ | KATLTVDKSSSTAYMQLSSL | WGTGTTVT |
| ASVKLSCKAS | GLEWIG | TSEDSAVYYCAN | VS |
| (SEQ ID NO: 66) | (SEQ ID NO: | (SEQ ID NO: 83) | (SEQ ID NO: |
| | 74) | | 93) |

V_{L} FR Sequences

| FR1 | FR2 | FR3 | FR4 |
|---|---|---|---|
| DIVMTQSQKFMSTSVG | WYQQKPGQ | GVPDRFTGSGSGTDFTLTIS | FGGGTKLEI |
| DRVSVTC | SPKALIY | NVQSEDLAEYFC | KR |
| (SEQ ID NO: 68) | (SEQ ID NO: | (SEQ ID NO: 84) | (SEQ ID NO: |
| | 75) | | 94) |
| QIVLTQSPAIMSASPG | WYQQKPGTS | GVPARFSGSGSGTSYSLTIS | FGAGTKLE |
| EKVTMTC | PKRWIY | SMEAEDAATYYC | LK |
| (SEQ ID NO: 69) | (SEQ ID NO: | (SEQ ID NO: 85) | (SEQ ID NO: |
| | 76) | | 95) |
| DIQMTRHPSSLSASMG | WLQQKPDGT | GVPKRFSGSRSGSDYSLTIS | FGGGTKLEI |
| ERVSLTC | IKRLIY | SLESEDFADYYC | (SEQ ID NO: |
| (SEQ ID NO: 70) | (SEQ ID NO: | (SEQ ID NO: 86) | 96) |
| | 77) | | |
| DVLMTQTPLSLPVSLG | WYLQKPGQS | GVPDRFSGSGSGTDFTLKIS | FGGGTKLEI |
| DQASISC | PKLLIY | RVEAEDLGVYYC | KRADA |
| (SEQ ID NO: 71) | (SEQ ID NO: | (SEQ ID NO: 87) | (SEQ ID NO: |
| | 78) | | 97) |
| QIVLTQSPAIMSASPG | WFQQKPGTS | GVPARFSGSGSGTSYSLTIS | FGGGTKLEI |
| EKVTITC | PKLWIY | RMEAEDAATYYC | KRAD |
| (SEQ ID NO: 72) | (SEQ ID NO: | (SEQ ID NO: 88) | (SEQ ID NO: |
| | 79) | | 98) |
| DIVLTQSPASLAVSLG | WNQQKPGQ | GVPARFSGSGSGTDFTLNIH | FGRGDQS |
| QRATISYRAS | PPRLLIY | PVEEEDAATYYC | WK |
| (SEQ ID NO: 73) | (SEQ ID NO: | (SEQ ID NO: 89) | (SEQ ID NO: |
| | 80) | | 99) |

TABLE 5
Amino Acid Sequences of Leader Sequences

MGWSYIILFLVATATGVHS
(SEQ ID NO: 100)
MGWSCIMLFLVATATGVHS
(SEQ ID NO: 101)
MGWSCIMFFLVATATGVHS
(SEQ ID NO: 102)
MEWSWVILILVAAATGVHS
(SEQ ID NO: 103)
MKTQTQVFVYMLLWLSGVDG
(SEQ ID NO: 104)
MDLQVQIFSFLLISASVILSRG
(SEQ ID NO: 105)
MVLISLLFWFPGTRC
(SEQ ID NO: 106)
MKMPVRLLVLMFWIPASSS
(SEQ ID NO: 107)
MDFQVQIFSFLLISASVIMSRG
(SEQ ID NO: 108)
METDTLLLWVLLLWVPGSTG
(SEQ ID NO: 109)

In certain embodiments, the amino acid sequence of the binding domain comprises a V_{H} sequence comprising a CDR1 selected from SEQ ID NOs: 37-39, a CDR2 selected from SEQ ID NOs: 46-47, or a CDR3 selected from SEQ ID NOs: 54-59.

In certain embodiments, the amino acid sequence of the binding domain comprises a V_{H} sequence comprising a CDR1 selected from SEQ ID NOs: 37-39, a CDR2 selected from SEQ ID NOs: 46-47, and a CDR3 selected from SEQ ID NOs: 54-59.

In certain embodiments, the amino acid sequence of the binding domain comprises a V_{H} sequence comprising a combination of CDR1, CDR2, and CDR3 included in Table 8. Each row of Table 8 illustrates a specific combination of CDR1, CDR2, and CDR3. For instance, the V_{H} sequence comprises CDR1 set forth by SEQ ID NO: 37, CDR2 set forth by SEQ ID NO: 46, and CDR3 set forth by SEQ ID NO: 54.

In certain embodiments, the amino acid sequence of the binding domain comprises a V_{H} sequence comprising an FR1 selected from SEQ ID NOs: 66-67, an FR2 represented by SEQ ID NO: 74, an FR3 selected from SEQ ID NOs: 81-83, or an FR4 selected from SEQ ID NOs: 90-93.

In certain embodiments, the amino acid sequence of the binding domain comprises a V_{H} sequence comprising an FR1 selected from SEQ ID NOs: 66-67, an FR2 represented by SEQ ID NO: 74, an FR3 selected from SEQ ID NOs: 81-83, and an FR4 selected from SEQ ID NOs: 90-93.

In certain embodiments, the amino acid sequence of the binding domain comprises a V_{L} sequence comprising a CDR1 selected from SEQ ID NOs: 40-45, a CDR2 selected from SEQ ID NOs: 48-53, or a CDR3 selected from SEQ ID NOs: 60-65.

In certain embodiments, the amino acid sequence of the binding domain comprises a V_{L} sequence comprising a CDR1 selected from SEQ ID NOs: 40-45, a CDR2 selected from SEQ ID NOs: 48-53, and a CDR3 selected from SEQ ID NOs: 60-65.

In certain embodiments, the amino acid sequence of the binding domain comprises a V_{L} sequence comprising a combination of CDR1, CDR2, and CDR3 included in Table 8. Each row of Table 8 illustrates a specific combination of CDR1, CDR2, and CDR3. For instance, the V_{L} sequence comprises CDR1 set forth by SEQ ID NO: 40, CDR2 set forth by SEQ ID NO: 48, and CDR3 set forth by SEQ ID NO: 60.

In certain embodiments, the amino acid sequence of the binding domain comprises a V_{L} sequence comprising an FR1 selected from SEQ ID NOs: 68-73, an FR2 selected from SEQ ID NOs: 75-80, an FR3 selected from SEQ ID NOs: 84-89, or an FR4 selected from SEQ ID NOs: 94-99.

In certain embodiments, the amino acid sequence of the binding domain comprises a V_{L} sequence comprising an FR1 selected from SEQ ID NOs: 68-73, an FR2 selected from SEQ ID NOs: 75-80, an FR3 selected from SEQ ID NOs: 84-89, and an FR4 selected from SEQ ID NOs: 94-99.

In certain embodiments, the amino acid sequence of the binding domain comprises a V_{H} sequence comprising a CDR1 selected from SEQ ID NOs: 37-39, a CDR2 selected from SEQ ID NOs: 46-47, and a CDR3 selected from SEQ ID NOs: 54-59; and a V_{L} sequence comprising a CDR1 selected from SEQ ID NOs: 40-45, a CDR2 selected from SEQ ID NOs: 48-53, and a CDR3 selected from SEQ ID NOs: 60-65. In some embodiments, the V_{H} sequence further comprises an FR1 selected from SEQ ID NOs: 66-67, an FR2 represented by SEQ ID NO: 74, an FR3 selected from SEQ ID NOs: 81-83, and an FR4 selected from SEQ ID NOs: 90-93. In some embodiments, the V_{L} sequence further comprises an FR1 selected from SEQ ID NOs: 68-73, an FR2 selected from SEQ ID NOs: 75-80, an FR3 selected from SEQ ID NOs: 84-89, and an FR4 selected from SEQ ID NOs: 94-99. In some embodiments, the V_{H} sequence further comprises a leader selected from SEQ ID NOs: 100-103. In some embodiments, the V_{L} sequence further comprises a leader selected from SEQ ID NOs: 104-109.

This disclosure also encompasses a binding domain comprises various combinations of V_{H} and V_{L} sequences disclosed above. Each V_{H} sequence disclosed above can be combined with various sequences of V_{L}s disclosed above. It is within the purview of one of ordinary skill in the art to select a V_{H} sequence and a V_{L} sequence based on the disclosure in this document to obtain a binding domain that specifically binds to at least a portion of B7-H3.

In certain embodiments, the binding domain comprises an scFv comprising a V_{H} domain and a V_{L} domain. The amino acid sequences of the V_{H} domain and the V_{L} domain are shown in Table 6 below. For each sequence, the FR1-4 sequences are shown underlined and the CDR1-3 sequences are shown in bold. In some embodiments, the binding domain comprises an scFv comprising a V_{H} domain having an amino acid sequence selected from SEQ ID NOs: 110-115 and a V_{L} domain having an amino acid sequence selected from SEQ ID NOs: 116-121.

For example, the scFv comprises a V_{H} domain having an amino acid sequence represented by SEQ ID NO: 110 and a V_{L} domain having an amino acid sequence represented by SEQ ID NO: 116; a V_{H} domain having an amino acid sequence represented by SEQ ID NO: 110 and a V_{L} domain having an amino acid sequence represented by SEQ ID NO: 117; a V_{H} domain having an amino acid sequence represented by SEQ ID NO: 110 and a V_{L} domain having an amino acid sequence represented by SEQ ID NO: 118; a V_{H} domain having an amino acid sequence represented by SEQ ID NO: 110 and a V_{L} domain having an amino acid sequence represented by SEQ ID NO: 119; a V_{H} domain having an amino acid sequence represented by SEQ ID NO: 110 and a V_{L} domain having an amino acid sequence represented by SEQ ID NO: 120; a V_{H} domain having an amino acid sequence represented by SEQ ID NO: 110 and a V_{L} domain having an amino acid sequence represented by SEQ ID NO: 121; a V_{H} domain having an amino acid sequence represented by SEQ ID NO: 111 and a V_{L} domain having an amino acid sequence represented by SEQ ID NO: 116; a V_{H} domain having an amino acid sequence represented by SEQ ID NO: 111 and a V_{L} domain having an amino acid sequence represented by SEQ ID NO: 117; a V_{H} domain having an amino acid sequence represented by SEQ ID NO: 111 and a V_{L} domain having an amino acid sequence represented by SEQ ID NO: 118; a V_{H} domain having an amino acid sequence represented by SEQ ID NO: 111 and a V_{L} domain having an amino acid sequence represented by SEQ ID NO: 119; a V_{H} domain having an amino acid sequence represented by SEQ ID NO: 111 and a V_{L} domain having an amino acid sequence represented by SEQ ID NO: 120; a V_{H} domain having an amino acid sequence represented by SEQ ID NO: 111 and a V_{L} domain having an amino acid sequence represented by SEQ ID NO: 121; a V_{H} domain having an amino acid sequence represented by SEQ ID NO: 112 and a V_{L} domain having an amino acid sequence represented by SEQ ID NO: 116; a V_{H} domain having an amino acid sequence represented by SEQ ID NO: 112 and a V_{L} domain having an amino acid sequence represented by SEQ ID NO: 117; a V_{H} domain having an amino acid sequence represented by SEQ ID NO: 112 and a V_{L} domain having an amino acid sequence represented by SEQ ID NO: 118; a V_{H} domain having an amino acid sequence represented by SEQ ID NO: 112 and a V_{L} domain having an amino acid sequence represented by SEQ ID NO: 119; a V_{H} domain having an amino acid sequence represented by SEQ ID NO: 112 and a V_{L} domain having an amino acid sequence represented by SEQ ID NO: 120; a V_{H} domain having an amino acid sequence represented by SEQ ID NO: 112 and a V_{L} domain having an amino acid sequence represented by SEQ ID NO: 121; a V_{H} domain having an amino acid sequence represented by SEQ ID NO: 113 and a V_{L} domain having an amino acid sequence represented by SEQ ID NO: 116; a V_{H} domain having an amino acid sequence represented by SEQ ID NO: 113 and a V_{L} domain having an amino acid sequence represented by SEQ ID NO: 117; a V_{H} domain having an amino acid sequence represented by SEQ ID NO: 113 and a V_{L} domain having an amino acid sequence represented by SEQ ID NO: 118; a V_{H} domain having an amino acid sequence represented by SEQ ID NO: 113 and a V_{L} domain having an amino acid sequence represented by SEQ ID NO: 119; a V_{H} domain having an amino acid sequence represented by SEQ ID NO: 113 and a V_{L} domain having an amino acid sequence represented by SEQ ID NO: 120; a V_{H} domain having an amino acid sequence represented by SEQ ID NO: 113 and a V_{L} domain having an amino acid sequence represented by SEQ ID NO: 121; a V_{H} domain having an amino acid sequence represented by SEQ ID NO: 114 and a V_{L} domain having an amino acid sequence represented by SEQ ID NO: 116; a V_{H} domain having an amino acid sequence represented by SEQ ID NO: 114 and a V_{L} domain having an amino acid sequence represented by SEQ ID NO: 117; a V_{H} domain having an amino acid sequence represented by SEQ ID NO: 114 and a V_{L} domain having an amino acid sequence represented by SEQ ID NO: 118; a V_{H} domain having an amino acid sequence represented by SEQ ID NO: 114 and a V_{L} domain having an amino acid sequence represented by SEQ ID NO: 119; a V_{H} domain having an amino acid sequence represented by SEQ ID NO: 114 and a V_{L} domain having an amino acid sequence represented by SEQ ID NO: 120; a V_{H} domain having an amino acid sequence represented by SEQ ID NO: 114 and a V_{L} domain having an amino acid sequence represented by SEQ ID NO: 121; a V_{H} domain having an amino acid sequence represented by SEQ ID NO: 115 and a V_{L} domain having an amino acid sequence represented by SEQ ID NO: 116; a V_{H} domain having an amino acid sequence represented by SEQ ID NO: 115 and a V_{L} domain having an amino acid sequence represented by SEQ ID NO: 117; a V_{H} domain having an amino acid sequence represented by SEQ ID NO: 115 and a V_{L} domain having an amino acid sequence represented by SEQ ID NO: 118; a V_{H} domain having an amino acid sequence represented by SEQ ID NO: 115 and a V_{L} domain having an amino acid sequence represented by SEQ ID NO: 119; a V_{H} domain having an amino acid sequence represented by SEQ ID NO: 115 and a V_{L} domain having an amino acid sequence represented by SEQ ID NO: 120; or a V_{H} domain having an amino acid sequence represented by SEQ ID NO: 115 and a V_{L} domain having an amino acid sequence represented by SEQ ID NO: 121.

TABLE 6
Amino Acid Sequences of VH and VL Chains

| V_{H} chain | V_{L} chain |
|---|---|
| (including CDRs 1-3 and FRs 1-4) | (including CDRs 1-3 and FRs 1-4) |
| QVQLQQPGAELVKPGASVKLSCKASGYT | DIVMTQSQKFMSTSVGDRVSVTCKASQ |
| FTSYWMHWVKQRPGQGLEWIGMIHPNS | NVGTNVAWYQQKPGQSPKALIYSASYR |
| GSTNYNEKFKSKATLTVDKSSSTAYMQL | YSGVPDRFTGSGSGTDFTLTISNVQSED |
| SSLTSEDSAVYYCARGGYFDYWGQGTT | LAEYFCQQYNSYPYTFGGGTKLEIKR |
| LTVSS | (SEQ ID NO: 116) |
| (SEQ ID NO: 110) | |
| QVQLQQPGAELVKPGASVKLSCKASGYT | QIVLTQSPAIMSASPGEKVTMTCSASSSI |
| FTSYWMHWVKQRPGQGLEWIGMIHPNS | SYMHWYQQKPGTSPKRWIYDTSKLASG |
| GSTNYNEKFKSKATLTVDKSSSTAYMQL | VPARFSGSGSGTSYSLTISSMEAEDAAT |
| SSLTSEDSAVYYCARESRQLDYWGQGT | YYCHQRSSYPLTFGAGTKLELK |
| TLTVSS | (SEQ ID NO: 117) |
| (SEQ ID NO: 111) | |
| QVQLQQPGAELVKPGASVKLSCKASGYT | DIQMTRHPSSLSASMGERVSLTCRASQE |
| FTNYWMHWVKQRPGQGLEWIGMIHPNS | ISGYLSWLQQKPDGTIKRLIYAASTLDSG |
| GSTNYNEKFKSKATLTVDKSSSTAYMQL | VPKRFSGSRSGSDYSLTISSLESEDFADY |
| SSLTSEDSAVYYCARRGDGDYWGQGTT | YCLQYASYPYTFGGGTKLEI |
| LTVSS | (SEQ ID NO: 118) |
| (SEQ ID NO: 112) | |
| QVQLQQPGAELVKPGASVKMSCKASGY | DVLMTQTPLSLPVSLGDQASISCRSSQSI |
| TFTSYWITWVKQRPGQGLEWIGDIYPGS | VHSNGNTYLEWYLQKPGQSPKLLIYKVS |
| GSTNYNEKFKSKATLTVDTSSSTAYMQL | NRFSGVPDRFSGSGSGTDFTLKISRVEA |
| SSLTSEDSAVYYCARGGTRFSPFAYWG | EDLGVYYCFQGSHVPWTFGGGTKLEIKR |
| QGTLVTVSAA | ADA |
| (SEQ ID NO: 113) | (SEQ ID NO: 119) |
| QVQLQQPGAELVKPGASVKLSCKASGYT | QIVLTQSPAIMSASPGEKVTITCSASSSVS |
| FTSYWMHWVKQRPGQGLEWIGMIHPNS | YMHWFQQKPGTSPKLWIYSTSNLASGV |
| GSTNYNEKFKSKATLTVDKSSSTAYMQL | PARFSGSGSGTSYSLTISRMEAEDAATY |
| SSLTSEDSAVYYCARYYYGSSYAMDYW | YCQQRSSYPYTFGGGTKLEIKRAD |
| GQGTSVTVS | (SEQ ID NO: 120) |
| (SEQ ID NO: 114) | |
| QVQLQQPGAELVKPGASVKLSCKASGYT | DIVLTQSPASLAVSLGQRATISYRASKSV |
| FTSYWMHWVKQRPGQGLEWIGMIHPNS | STSGYSYMHWNQQKPGQPPRLLIYLVSN |
| GSTNYNEKFKSKATLTVDKSSSTAYMQL | LESGVPARFSGSGSGTDFTLNIHPVEEE |
| SSLTSEDSAVYYCANYYGTNVWGTGTTV | DAATYYCQHIREAYTFGRGDQSWK |
| TVS | (SEQ ID NO: 121) |
| (SEQ ID NO: 115) | |

### Uses

The present disclosure also provides methods for treating a disease or condition, wherein the methods comprise administering to a subject in need thereof an effective amount of a host cell, composition, or unit dose of the present disclosure, wherein the disease or condition expresses or is otherwise associated with the antigen that is specifically bound by the CAR. In certain embodiments, the disease or condition is a hyperproliferative or proliferative disease, such as a cancer, an autoimmune disease, or an infectious disease (e.g., viral, bacterial, fungal, or parasitic).

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a CAR as described herein is administered intravenously, intraperitoneally, intratumorally, into the bone marrow, into a lymph node, or into the cerebrospinal fluid so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a CAR of the present disclosure, or a composition comprising the host cell. The amount of cells in a composition is at least one cell (for example, one CAR-modified CD8+ T cell subpopulation; one CAR-modified CD4+ T cell subpopulation; one CAR-modified NK cell subpopulation) or is more typically greater than 10² cells, for example, up to 10⁶, up to 10⁷, up to 10⁸ cells, up to 10⁹ cells, or 10¹⁰ cells or more, such as about 10¹¹ cells/m². In certain embodiments, the cells are administered in a range from about 10⁵ to about 10¹¹ cells/m², preferably in a range of about 10⁵ or about 10⁶ to about 109 or about 10¹⁰ cells/m². The number of cells will depend upon the ultimate use for which the composition is intended as well as the type of cells included therein. For example, cells modified to contain a CAR specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mLs or less, 250 mLs or less, or 100 mLs or less. In embodiments, the density of the desired cells is typically greater than 10⁴ cells/mL and generally is greater than 10⁷ cells/mL, generally 10⁸ cells/mL or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In any of the presently disclosed embodiments, the host cell is an allogeneic cell, a syngeneic cell, or an autologous cell.

Unit doses are also provided herein which comprise a host cell (e.g., a modified immune cell comprising a polynucleotide of the present disclosure) or host cell composition of this disclosure. In some embodiments, a unit dose comprises (i) a composition comprising at least about 50% modified CD4+ T cells, combined with (ii) a composition comprising at least about 50% modified CD8+ T cells, in about a 1:1 ratio, wherein the unit dose contains a reduced amount or substantially no naïve T cells.

Also contemplated are pharmaceutical compositions that comprise cells expressing the CARs as disclosed herein and a pharmaceutically acceptable carrier, diluents, or excipient. Suitable excipients include water, saline, dextrose, glycerol, or the like and combinations thereof. In embodiments, compositions comprising host cells as disclosed herein further comprise a suitable infusion media.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, NK cells) in order to administer a desired, CAR-expressing T-cell composition to a subject as an adoptive immunotherapy. In certain embodiments, the host cell is a hematopoietic progenitor cell or a human immune cell. In certain embodiments, the immune system cell is a CD4+ T cell, a CD8+ T cell, a CD4− CD8− double-negative T cell, an NK cell, or any combination thereof. In certain embodiments, the immune system cell is a naïve T cell, a central memory T cell, a stem cell memory T cell, an effector memory T cell, an NK cell, or any combination thereof. In particular embodiments, the cell is a CD4+ T cell. In particular embodiments, the cell is a CD8+ T cell. In particular embodiments, the cell is an NK cell.

As used herein, administration of a composition refers to delivering the same to a subject, regardless of the route or mode of delivery. Administration may be affected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., CAR-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., host cell, CAR, unit dose, or composition) refers to an amount sufficient, at dosages and for periods of time needed, to achieve the desired clinical results or beneficial treatment, as described herein. An effective amount may be delivered in one or more administrations.

Methods according to this disclosure may further include administering one or more additional agents to treat the disease or disorder in a combination therapy. For example, in certain embodiments, a combination therapy comprises administering a CAR (or an engineered host cell expressing the same) with (concurrently, simultaneously, or sequentially) an immune checkpoint inhibitor. In some embodiments, a combination therapy comprises administering CAR of the present disclosure (or an engineered host cell expressing the same) with an agonist of a stimulatory immune checkpoint agent. In further embodiments, a combination therapy comprises administering a CAR of the present disclosure (or an engineered host cell expressing the same) with a secondary therapy, such as chemotherapeutic agent, a radiation therapy, a surgery, an antibody, or any combination thereof.

Cytokines are used to manipulate host immune response towards anticancer activity (see, e.g., Floros & Tarhini, 2015). Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times. The headings provided herein are for convenience only and do not interpret the scope or meaning of the described technology.

Any patents, applications and other references cited herein are incorporated herein by reference. Aspects of the described technology can be modified, if necessary, to employ the systems, functions, and concepts of the various references described above to provide yet further embodiments.

These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

The foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### EXAMPLES

The following examples are illustrative of several embodiments of the present technology:

### Example 1: Generation of and Characterization of B7-H3 Monoclonal Antibodies

In this example, anti-B7-H3 monoclonal antibodies (mAbs) were generated, functionally tested, and sequenced. Portions of the V_{L} and V_{H} chains for each mAb were used to generate the CARs described in Example 2.

Generation of anti-B7-H3 mAbs. The functional domain for B7-H3 is IgV. Accordingly, a human B7-H3 IgV-Ig protein was generated by fusing the B7-H3 IgV coding region (amino acid residues E35-A139) to a human IgG1 Fc tag from plasmid pMT/BiP as previously described (Zhao, et al., 2013). The protein was expressed in an S2 system as previously described (Zhao, et al., 2013) and then purified. Mice were immunized with B7-H3 IgV-Ig protein and hybridomas were generated using standard techniques from splenocytes fused to NSO myeloma cells as previously described (Zhao, et al., 2013).

Characterizing mAbs 1G5, 15F9, 23B2, 8K12, 12B4, and 24D12. 1G5, 15F9, 81812, 124, and 24D12 are IgG3 mAbs having kappa chains, whereas 23B2 is an IgG1 mAb having a kappa chain. The binding affinities (K_{d}) of mAbs 1G5, 15F9, 231B2, 81B12, 1214, and 24D12 to human B7-H3 and to mouse B7-H3 protein were determined using standard techniques including by Surface Plasmon Resonance (SPM) and are shown in Table 7.

TABLE 7
Kinetic parameters from Surface
Plasmon Resonance of various mAbs

| mAb clone | Isotype | Antigen | Kd (nM) |
|---|---|---|---|
| 1G5 | IgG3, K | Human B7-H3 | 4.86 |
| 1G5 | IgG3, K | Mouse B7-H3 | 0.81 |
| 15F9 | IgG3, K | Human B7-H3 | 4.51 |
| 15F9 | IgG3, K | Mouse B7-H3 | 1.76 |
| 23B2 | IgG1, K | Human B7-H3 | 11.08 |
| 23B2 | IgG1, K | Mouse B7-H3 | 10.18 |
| 8B12 | IgG3, K | Human B7-H3 | 0.32 |
| 8B12 | IgG3, K | Mouse B7-H3 | 4.01 |
| 12B4 | IgG3, K | Human B7-H3 | 5.62 |
| 12B4 | IgG3, K | Mouse B7-H3 | 1.71 |
| 24D12 | IgG3, K | Human B7-H3 | 4.51 |
| 24D12 | IgG3, K | Mouse B7-H3 | 1.76 |

Sequencing mAbs 1G5, 15F9, 23B2, 8812, 12B4, and 24D12. The hybridomas 1G5, 15F9, 23B2, 8B12, 12B4, and 24D12 were sequenced using standard techniques. Each hybridoma has a unique V_{H} sequence and a unique V_{L} sequence (see SEQ ID NOs: 7-18 of Table 1 for amino acid sequences and SEQ ID NOs: 25-36 of Table 2 for nucleic acid sequences).

### Example 2: Generation of and Functional Testing of B7-H3 CAR-T Cells

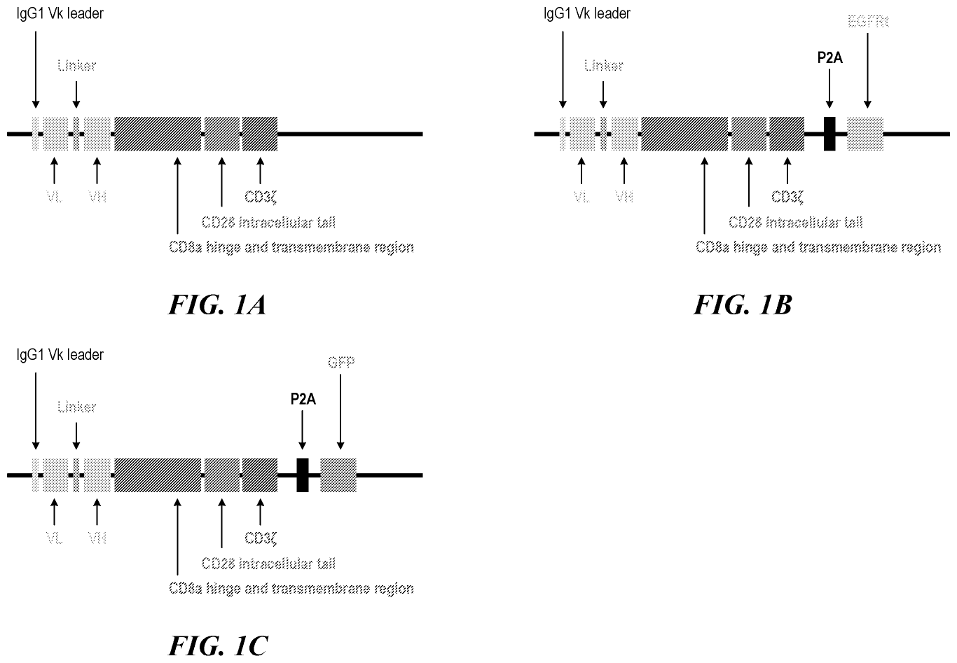

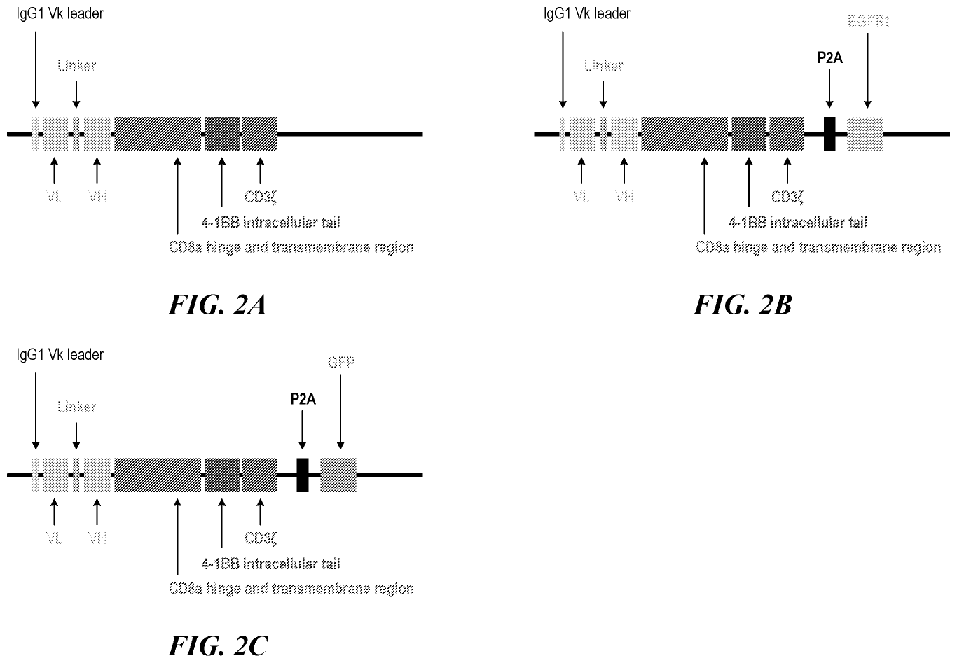

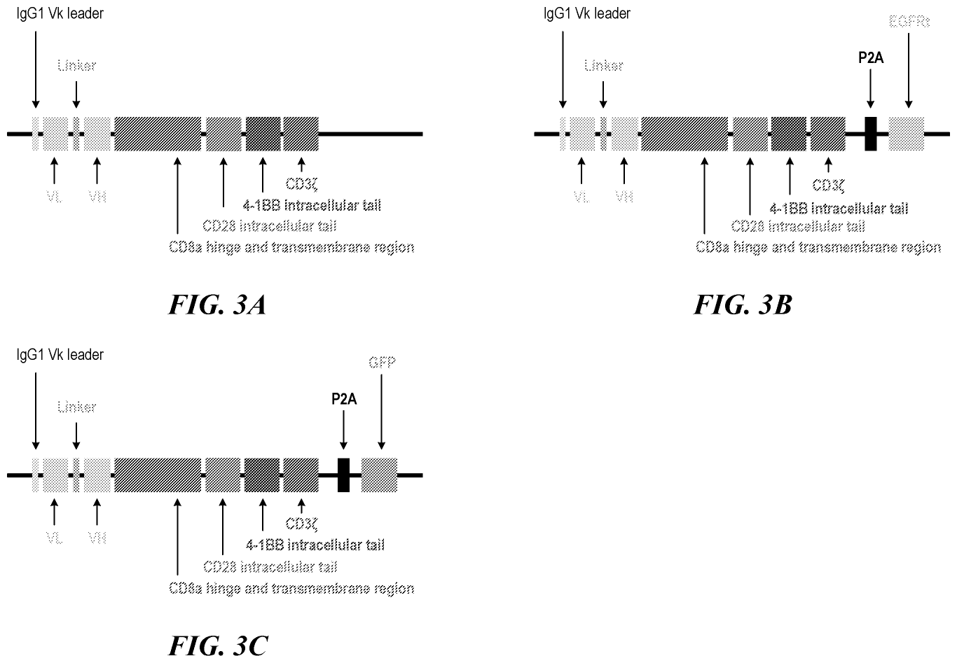

Generation of B7-H3 CAR-T viral vectors with mAb 1G5, 15F9, 23B2, 8812, 12B4, and 24D12 as scFvs. As shown in FIGS. 1-4, eighteen B7-H3 CAR-T vectors were generated, six for each mAb 1G5, 15F9, 23B2, 8B12, 12B4, or 24D12. Each of the eighteen vectors includes polynucleotides encoding the following polypeptides, schematically from left to right, a IgG1 V_{K} Leader sequence, a V_{L} domain (from 1 G5, 15F9, 23B2, 8B12, 12B4, or 24D12), a linker domain, a V_{H} domain from 1 G5, 15F9, 23B2, 8B12, 12B4, or 24D12), a CD8α hinge and CD8α transmembrane region, a CD28 intracellular tail, a 4-1 BB intracellular tail, and a CD3ζ domain. For all vectors, the mAb sequence used to generate the V_{L} domain is the same mAb from which the V_{H} sequence was derived. As shown, the B7-H3 CAR-T vector optionally includes GFP as a transduction marker. In this example, three vectors were generated for 1 G5 (one with EGFRt, one with GFP, and one without), three vectors were generated for 15F9 (one with EGFRt, one with GFP, and one without), three vectors were generated for 23B2 (one with EGFRt, one with GFP, and one without), three vectors were generated for 8B12 (one with EGFRt, one with GFP, and one without), three vectors were generated for 12B4 (one with EGFRt, one with GFP, and one without), and three vectors were generated for 24D12 (one with EGFRt, one with GFP, and one without). These eighteen vectors are illustrated schematically by the two vectors shown in FIGS. 1-4.

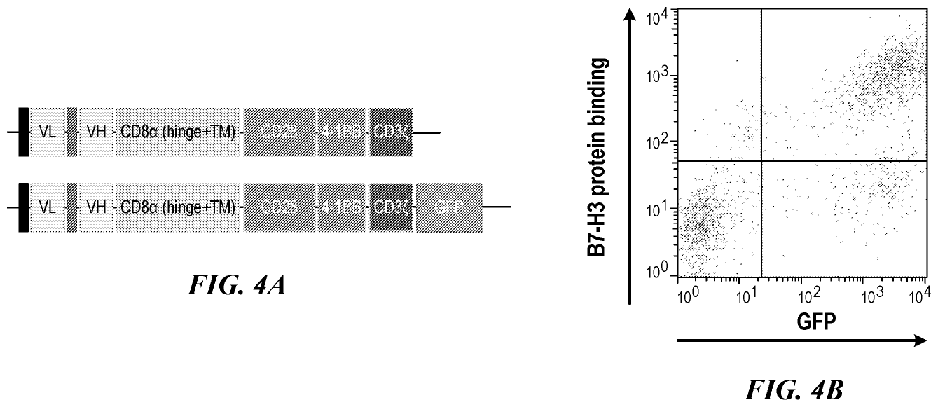

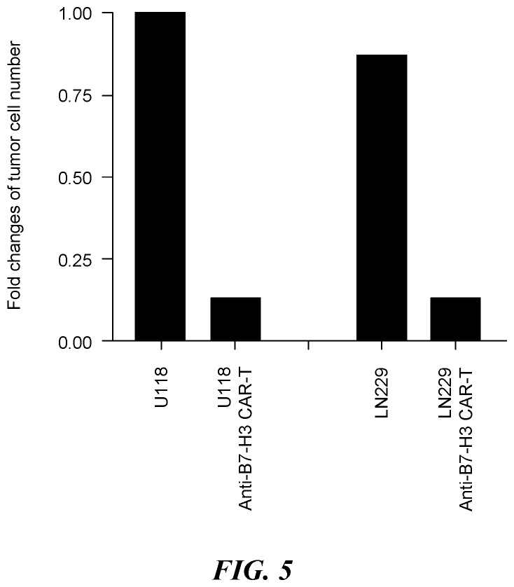

Human peripheral blood mononuclear cells (PBMCs) were transduced with each of the nine vectors. CAR positive T cells were sorted from the total PBMC population by fluorescence assisted cell sorting (FACS) since human T cells expressing the B7-H3 CAR illustrated in FIG. 4A were denoted by GFP positive T cells (GFP was used as a transduction marker) (FIG. 4B).

The B7-H3 CAR-T cells were mixed at a 1 to 1 ratio with normal T cells and then co-cultured with glioblastoma U118 and LB229 tumor cells at an effector: target ratio of 1:1. After five days, the co-cultures were analyzed by flow cytometry. The B7-H3 CAR-T cells are functional as they killed the majority of U118 or LB229 tumor cells (FIG., 5).

### Example 3: Expression of B7-H3 CAR-T in Human Primary T Cells

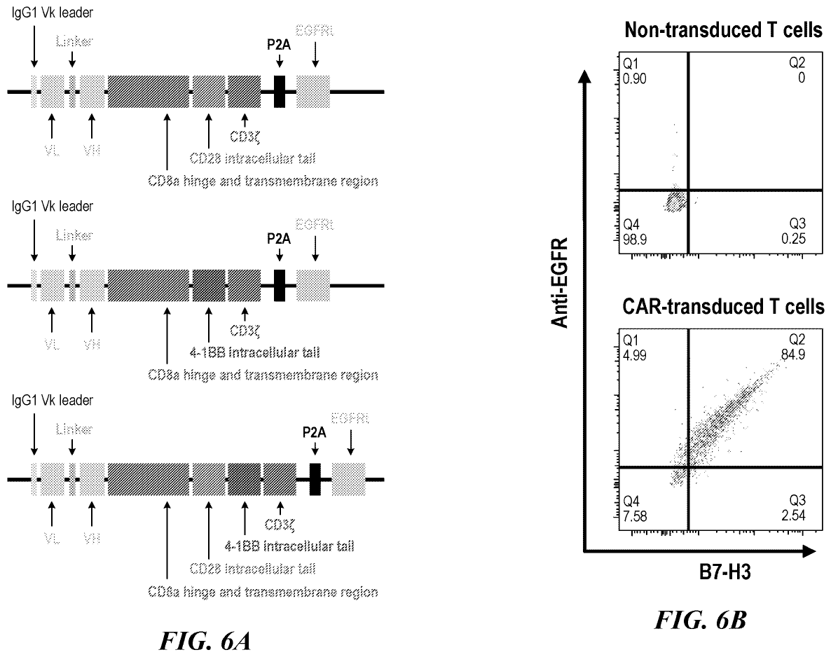

FIG. 6 illustrates that six different anti-B7-H3 CAR-T vectors with six different anti-B7-H3 mAbs (1G5, 15F9, 23B2, 8B12, 12B4, and 24D12) were generated and used to transduce human primary T cells. The transduced human primary T cells expressed EGFR and recognized B7-H3 antigen; whereas non-transduced human primary T cells did not express any EGFR or recognize B7-H3 antigen, as shown by FACS (FIG. 6B).

From the foregoing, it will be appreciated that specific embodiments of the present disclosure have been described herein for purposes of illustration, but that various modifications may be made without deviating from the scope of the present disclosure. Accordingly, the present disclosure is not limited except as by the appended claims.

### REFERENCES

1. Baral et al. B7-H3 and B7-H1 expression in cerebral spinal fluid and tumor tissue correlates with the malignancy grade of glioma patients. Oncol Lett 8:1195-1201 (2014)
2. Brentjens et al. Genetically targeted T cells eradicate systemic acute lymphoblastic leukemia xenografts. Clin Cancer Res 13(18 Pt 1):5426-5435 (2007)
3. Chen et al. The coexpression and clinical significance of costimulatory molecules B7-H1, B7-H3, and B7-H4 in human pancreatic cancer. Onco Targets Ther 7:1465-1472 (2014).
4. Dossett et al. Adoptive immunotherapy of disseminated leukemia with TCR-transduced, CD8⁺ T cells expressing a known endogenous TCR. Mol Ther 17(4):742-749 (2009)
5. Floros & Tarhini. Anticancer Cytokines: Biology and Clinical Effects of Interferon-α2, Interleukin (IL)-2, IL-15, IL-21, and IL-12. Semin Oncol 42(4):539-548 (2015)
6. Guest et al. The role of extracellular spacer regions in the optimal design of chimeric immune receptors: evaluation of four different scFvs and antigens. J Immunother 28(3):203-211 (2005)
7. Harris & Kranz. Adoptive T Cell Therapies: A Comparison of T Cell Receptors and Chimeric Antigen Receptors. Trends Pharmacol Sci 37(3):220-230 (2016)
8. Hu et al. Expression of costimulatory molecule B7-H3 and its prognostic implications in human acute leukemia. Hematology 20:187-195 (2015)
9. Ingebrigtsen et al. B7-H3 expression in colorectal cancer: associations with clinicopathological parameters and patient outcome. BMC Cancer 14:602 (2014).
10. Kuball et al. Facilitating matched pairing and expression of TCR chains introduced into human T cells. Blood 109(6):2331-2338 (2007)
11. Leen et al. Improving T cell therapy for cancer. Annu Rev Immunol 25:243-265 (2007)
12. Patel et al. Impact of chimeric immune receptor extracellular protein domains on T cell function. Gene Therapy 6(3):412-419 (1999)
13. Picarda et al. Molecular Pathways: Targeting B7-H3 (CD276) for Human Cancer Immunotherapy. Clin Cancer Res 22:3425-3431 (2016)
14. Qin et al. B7-H3 is a new cancer-specific endothelial marker in clear cell renal cell carcinoma. Onco Targets Ther 6:1667-1673 (2013)
15. Sadelain et al. The basic principles of chimeric antigen receptor design. Cancer Discov 3(4):388-398 (2013)
16. Scatchard et al. The attractions of proteins for small molecules and ions. Ann. N.Y. Acad. Sci. 51:660 (1949)
16. Schmitt et al. T cell receptor gene therapy for cancer. Hum Gene Ther 20(11):1240-1248 (2009)
17. Scholten et al. Codon modification of T cell receptors allows enhanced functional expression in transgenic human T cells. Clin Immunol 119(2):135-145 (2006)
18. Stone et al. A novel T cell receptor single-chain signaling complex mediates antigen-specific T cell activity and tumor control. Cancer Immunol Immunother 63(11):1163-1176 (2014)
19. Sun et al. B7-H3 and B7-H4 expression in non-small-cell lung cancer. Lung Cancer 53:143-151 (2006)
20. Sun et al. B7-H3 is expressed in human hepatocellular carcinoma and is associated with tumor aggressiveness and postoperative recurrence. Cancer Immunol Immunother 61:2171-2182 (2012)
21. Sun et al. B7-H3 expression in breast cancer and upregulation of VEGF through gene silence. Onco Targets Ther 7:1979-1986 (2014)
22. Till et al. Adoptive immunotherapy for indolent non-Hodgkin lymphoma and mantle cell lymphoma using genetically modified autologous CD20-specific T cells. Blood 112(6):2261-2271 (2008)
23. Vigdorovich et al. Structure and T cell inhibition properties of B7 family member, B7-H3. Structure 21:707-717 (2013)
24. Walseng et al. A TCR-based Chimeric Antigen Receptor. Sci Rep 7(1):10713 (2017)
25. Wang et al. Optimizing adoptive polyclonal T cell immunotherapy of lymphomas, using a chimeric T cell receptor possessing CD28 and CD137 costimulatory domains. Hum Gene Ther 18:712-725 (2007)
26. Wang et al. B7-H3 associated with tumor progression and epigenetic regulatory activity in cutaneous melanoma. J Invest Dermatol 133:2050-2058 (2013)
27. Wang et al. B7-H3 is overexpressed in patients suffering osteosarcoma and associated with tumor aggressiveness and metastasis. PLoS One 8:e70689 (2013)
28. Wilson. Tech.Sight. Analyzing biomolecular interactions. Science 295(5562):2103-2105 (2002)
29. Wolff et al. Monoclonal antibody homodimers: enhanced antitumor activity in nude mice. Cancer Res 53(11):2560-2565 (1993)
30. Xylinas et al. Association of T-cell co-regulatory protein expression with clinical outcomes following radical cystectomy for urothelial carcinoma of the bladder. Eur J Surg Oncol 40:121-127 (2014)
31. Zang & Allison. The B7 family and cancer therapy: costimulation and coinhibition. Clin Cancer Res 13:5271-5279 (2007)
32. Zang et al. B7-H3 and B7x are highly expressed in human prostate cancer and associated with disease spread and poor outcome. Proc Natl Acad Sci USA 104:19458-19463 (2007)
33. Zang et al. Tumor associated endothelial expression of B7-H3 predicts survival in ovarian carcinomas. Mod Pathol 23:1104-1112 (2010)
34. Zhao et al. HHLA2 is a member of the B7 family and inhibits human CD4 and CD8 T-cell function. Proc Natl Acad Sci USA 110:9879-9884 (2013)

TABLE 8
Amino Acid Sequences of CDRs
V_{H} CDR Sequences

| CDR1 | CDR2 | CDR3 |
|---|---|---|
| GYTFTSYWMH | MIHPNSGSTNYNEKFKS | GGYFDY |
| (SEQ ID NO: 37) | (SEQ ID NO: 46) | (SEQ ID NO: 54) |
| GYTFTSYWMH | MIHPNSGSTNYNEKFKS | ARESRQLDY |
| (SEQ ID NO: 37) | (SEQ ID NO: 46) | (SEQ ID NO: 55) |
| GYTFTSYWMH | MIHPNSGSTNYNEKFKS | RGDGDY |
| (SEQ ID NO: 37) | (SEQ ID NO: 46) | (SEQ ID NO: 56) |
| GYTFTSYWMH | MIHPNSGSTNYNEKFKS | ARGGTRFSPFAY |
| (SEQ ID NO: 37) | (SEQ ID NO: 46) | (SEQ ID NO: 57) |
| GYTFTSYWMH | MIHPNSGSTNYNEKFKS | YYYGSSYAMDY |
| (SEQ ID NO: 37) | (SEQ ID NO: 46) | (SEQ ID NO: 58) |
| GYTFTSYWMH | MIHPNSGSTNYNEKFKS | YYGTNV |
| (SEQ ID NO: 37) | (SEQ ID NO: 46) | (SEQ ID NO: 59) |
| GYTFTSYWMH | DIYPGSGSTNYNEKFKS | GGYFDY |
| (SEQ ID NO: 37) | (SEQ ID NO: 47) | (SEQ ID NO: 54) |
| GYTFTSYWMH | DIYPGSGSTNYNEKFKS | ARESRQLDY |
| (SEQ ID NO: 37) | (SEQ ID NO: 47) | (SEQ ID NO: 55) |
| GYTFTSYWMH | DIYPGSGSTNYNEKFKS | RGDGDY |
| (SEQ ID NO: 37) | (SEQ ID NO: 47) | (SEQ ID NO: 56) |
| GYTFTSYWMH | DIYPGSGSTNYNEKFKS | ARGGTRFSPFAY |
| (SEQ ID NO: 37) | (SEQ ID NO: 47) | (SEQ ID NO: 57) |
| GYTFTSYWMH | DIYPGSGSTNYNEKFKS | YYYGSSYAMDY |
| (SEQ ID NO: 37) | (SEQ ID NO: 47) | (SEQ ID NO: 58) |
| GYTFTSYWMH | DIYPGSGSTNYNEKFKS | YYGTNV |
| (SEQ ID NO: 37) | (SEQ ID NO: 47) | (SEQ ID NO: 59) |
| GYTFTNYWMH | MIHPNSGSTNYNEKFKS | GGYFDY |
| (SEQ ID NO: 38) | (SEQ ID NO: 46) | (SEQ ID NO: 54) |
| GYTFTNYWMH | MIHPNSGSTNYNEKFKS | ARESRQLDY |
| (SEQ ID NO: 38) | (SEQ ID NO: 46) | (SEQ ID NO: 55) |
| GYTFTNYWMH | MIHPNSGSTNYNEKFKS | RGDGDY |
| (SEQ ID NO: 38) | (SEQ ID NO: 46) | (SEQ ID NO: 56) |
| GYTFTNYWMH | MIHPNSGSTNYNEKFKS | ARGGTRFSPFAY |
| (SEQ ID NO: 38) | (SEQ ID NO: 46) | (SEQ ID NO: 57) |
| GYTFTNYWMH | MIHPNSGSTNYNEKFKS | YYYGSSYAMDY |
| (SEQ ID NO: 38) | (SEQ ID NO: 46) | (SEQ ID NO: 58) |
| GYTFTNYWMH | MIHPNSGSTNYNEKFKS | YYGTNV |
| (SEQ ID NO: 38) | (SEQ ID NO: 46) | (SEQ ID NO: 59) |
| GYTFTNYWMH | DIYPGSGSTNYNEKFKS | GGYFDY |
| (SEQ ID NO: 38) | (SEQ ID NO: 47) | (SEQ ID NO: 54) |
| GYTFTNYWMH | DIYPGSGSTNYNEKFKS | ARESRQLDY |
| (SEQ ID NO: 38) | (SEQ ID NO: 47) | (SEQ ID NO: 55) |
| GYTFTNYWMH | DIYPGSGSTNYNEKFKS | RGDGDY |
| (SEQ ID NO: 38) | (SEQ ID NO: 47) | (SEQ ID NO: 56) |
| GYTFTNYWMH | DIYPGSGSTNYNEKFKS | ARGGTRFSPFAY |
| (SEQ ID NO: 38) | (SEQ ID NO: 47) | (SEQ ID NO: 57) |
| GYTFTNYWMH | DIYPGSGSTNYNEKFKS | YYYGSSYAMDY |
| (SEQ ID NO: 38) | (SEQ ID NO: 47) | (SEQ ID NO: 58) |
| GYTFTNYWMH | DIYPGSGSTNYNEKFKS | YYGTNV |
| (SEQ ID NO: 38) | (SEQ ID NO: 47) | (SEQ ID NO: 59) |
| GYTFTSYWIT | MIHPNSGSTNYNEKFKS | GGYFDY |
| (SEQ ID NO: 39) | (SEQ ID NO: 46) | (SEQ ID NO: 54) |
| GYTFTSYWIT | MIHPNSGSTNYNEKFKS | ARESRQLDY |
| (SEQ ID NO: 39) | (SEQ ID NO: 46) | (SEQ ID NO: 55) |
| GYTFTSYWIT | MIHPNSGSTNYNEKFKS | RGDGDY |
| (SEQ ID NO: 39) | (SEQ ID NO: 46) | (SEQ ID NO: 56) |
| GYTFTSYWIT | MIHPNSGSTNYNEKFKS | ARGGTRFSPFAY |
| (SEQ ID NO: 39) | (SEQ ID NO: 46) | (SEQ ID NO: 57) |
| GYTFTSYWIT | MIHPNSGSTNYNEKFKS | YYYGSSYAMDY |
| (SEQ ID NO: 39) | (SEQ ID NO: 46) | (SEQ ID NO: 58) |
| GYTFTSYWIT | MIHPNSGSTNYNEKFKS | YYGTNV |
| (SEQ ID NO: 39) | (SEQ ID NO: 46) | (SEQ ID NO: 59) |
| GYTFTSYWIT | DIYPGSGSTNYNEKFKS | GGYFDY |
| (SEQ ID NO: 39) | (SEQ ID NO: 47) | (SEQ ID NO: 54) |
| GYTFTSYWIT | DIYPGSGSTNYNEKFKS | ARESRQLDY |
| (SEQ ID NO: 39) | (SEQ ID NO: 47) | (SEQ ID NO: 55) |
| GYTFTSYWIT | DIYPGSGSTNYNEKFKS | RGDGDY |
| (SEQ ID NO: 39) | (SEQ ID NO: 47) | (SEQ ID NO: 56) |
| GYTFTSYWIT | DIYPGSGSTNYNEKFKS | ARGGTRFSPFAY |
| (SEQ ID NO: 39) | (SEQ ID NO: 47) | (SEQ ID NO: 57) |
| GYTFTSYWIT | DIYPGSGSTNYNEKFKS | YYYGSSYAMDY |
| (SEQ ID NO: 39) | (SEQ ID NO: 47) | (SEQ ID NO: 58) |
| GYTFTSYWIT | DIYPGSGSTNYNEKFKS | YYGTNV |
| (SEQ ID NO: 39) | (SEQ ID NO: 47) | (SEQ ID NO: 59) |

V_{L} CDR Sequences

| CDR1 | CDR2 | CDR3 |
|---|---|---|
| KASQNVGTNVA | SASYRYS | QQYNSYPYT |
| (SEQ ID NO: 40) | (SEQ ID NO: 48) | (SEQ ID NO: 60) |
| KASQNVGTNVA | SASYRYS | HQRSSYPLT |
| (SEQ ID NO: 40) | (SEQ ID NO: 48) | (SEQ ID NO: 61) |
| KASQNVGTNVA | SASYRYS | LQYASYPYT |
| (SEQ ID NO: 40) | (SEQ ID NO: 48) | (SEQ ID NO: 62) |
| KASQNVGTNVA | SASYRYS | FQGSHVPWT |
| (SEQ ID NO: 40) | (SEQ ID NO: 48) | (SEQ ID NO: 63) |
| KASQNVGTNVA | SASYRYS | QQRSSYPYT |
| (SEQ ID NO: 40) | (SEQ ID NO: 48) | (SEQ ID NO: 64) |
| KASQNVGTNVA | SASYRYS | QHIREAYT |
| (SEQ ID NO: 40) | (SEQ ID NO: 48) | (SEQ ID NO: 65) |
| KASQNVGTNVA | DTSKLAS | QQYNSYPYT |
| (SEQ ID NO: 40) | (SEQ ID NO: 49) | (SEQ ID NO: 60) |
| KASQNVGTNVA | DTSKLAS | HQRSSYPLT |
| (SEQ ID NO: 40) | (SEQ ID NO: 49) | (SEQ ID NO: 61) |
| KASQNVGTNVA | DTSKLAS | LQYASYPYT |
| (SEQ ID NO: 40) | (SEQ ID NO: 49) | (SEQ ID NO: 62) |
| KASQNVGTNVA | DTSKLAS | FQGSHVPWT |
| (SEQ ID NO: 40) | (SEQ ID NO: 49) | (SEQ ID NO: 63) |
| KASQNVGTNVA | DTSKLAS | QQRSSYPYT |
| (SEQ ID NO: 40) | (SEQ ID NO: 49) | (SEQ ID NO: 64) |
| KASQNVGTNVA | DTSKLAS | QHIREAYT |
| (SEQ ID NO: 40) | (SEQ ID NO: 49) | (SEQ ID NO: 65) |
| KASQNVGTNVA | AASTLDS | QQYNSYPYT |
| (SEQ ID NO: 40) | (SEQ ID NO: 50) | (SEQ ID NO: 60) |
| KASQNVGTNVA | AASTLDS | HQRSSYPLT |
| (SEQ ID NO: 40) | (SEQ ID NO: 50) | (SEQ ID NO: 61) |
| KASQNVGTNVA | AASTLDS | LQYASYPYT |
| (SEQ ID NO: 40) | (SEQ ID NO: 50) | (SEQ ID NO: 62) |
| KASQNVGTNVA | AASTLDS | FQGSHVPWT |
| (SEQ ID NO: 40) | (SEQ ID NO: 50) | (SEQ ID NO: 63) |
| KASQNVGTNVA | AASTLDS | QQRSSYPYT |
| (SEQ ID NO: 40) | (SEQ ID NO: 50) | (SEQ ID NO: 64) |
| KASQNVGTNVA | AASTLDS | QHIREAYT |
| (SEQ ID NO: 40) | (SEQ ID NO: 50) | (SEQ ID NO: 65) |
| KASQNVGTNVA | KVSNRFS | QQYNSYPYT |
| (SEQ ID NO: 40) | (SEQ ID NO: 51) | (SEQ ID NO: 60) |
| KASQNVGTNVA | KVSNRFS | HQRSSYPLT |
| (SEQ ID NO: 40) | (SEQ ID NO: 51) | (SEQ ID NO: 61) |
| KASQNVGTNVA | KVSNRFS | LQYASYPYT |
| (SEQ ID NO: 40) | (SEQ ID NO: 51) | (SEQ ID NO: 62) |
| KASQNVGTNVA | KVSNRFS | FQGSHVPWT |
| (SEQ ID NO: 40) | (SEQ ID NO: 51) | (SEQ ID NO: 63) |
| KASQNVGTNVA | KVSNRFS | QQRSSYPYT |
| (SEQ ID NO: 40) | (SEQ ID NO: 51) | (SEQ ID NO: 64) |
| KASQNVGTNVA | KVSNRFS | QHIREAYT |
| (SEQ ID NO: 40) | (SEQ ID NO: 51) | (SEQ ID NO: 65) |
| KASQNVGTNVA | STSNLAS | QQYNSYPYT |
| (SEQ ID NO: 40) | (SEQ ID NO: 52) | (SEQ ID NO: 60) |
| KASQNVGTNVA | STSNLAS | HQRSSYPLT |
| (SEQ ID NO: 40) | (SEQ ID NO: 52) | (SEQ ID NO: 61) |
| KASQNVGTNVA | STSNLAS | LQYASYPYT |
| (SEQ ID NO: 40) | (SEQ ID NO: 52) | (SEQ ID NO: 62) |
| KASQNVGTNVA | STSNLAS | FQGSHVPWT |
| (SEQ ID NO: 40) | (SEQ ID NO: 52) | (SEQ ID NO: 63) |
| KASQNVGTNVA | STSNLAS | QQRSSYPYT |
| (SEQ ID NO: 40) | (SEQ ID NO: 52) | (SEQ ID NO: 64) |
| KASQNVGTNVA | STSNLAS | QHIREAYT |
| (SEQ ID NO: 40) | (SEQ ID NO: 52) | (SEQ ID NO: 65) |
| KASQNVGTNVA | LVSNLES | QQYNSYPYT |
| (SEQ ID NO: 40) | (SEQ ID NO: 53) | (SEQ ID NO: 60) |
| KASQNVGTNVA | LVSNLES | HQRSSYPLT |
| (SEQ ID NO: 40) | (SEQ ID NO: 53) | (SEQ ID NO: 61) |
| KASQNVGTNVA | LVSNLES | LQYASYPYT |
| (SEQ ID NO: 40) | (SEQ ID NO: 53) | (SEQ ID NO: 62) |
| KASQNVGTNVA | LVSNLES | FQGSHVPWT |
| (SEQ ID NO: 40) | (SEQ ID NO: 53) | (SEQ ID NO: 63) |
| KASQNVGTNVA | LVSNLES | QQRSSYPYT |
| (SEQ ID NO: 40) | (SEQ ID NO: 53) | (SEQ ID NO: 64) |
| KASQNVGTNVA | LVSNLES | QHIREAYT |
| (SEQ ID NO: 40) | (SEQ ID NO: 53) | (SEQ ID NO: 65) |
| SASSSISYMH | SASYRYS | QQYNSYPYT |
| (SEQ ID NO: 41) | (SEQ ID NO: 48) | (SEQ ID NO: 60) |
| SASSSISYMH | SASYRYS | HQRSSYPLT |
| (SEQ ID NO: 41) | (SEQ ID NO: 48) | (SEQ ID NO: 61) |
| SASSSISYMH | SASYRYS | LQYASYPYT |
| (SEQ ID NO: 41) | (SEQ ID NO: 48) | (SEQ ID NO: 62) |
| SASSSISYMH | SASYRYS | FQGSHVPWT |
| (SEQ ID NO: 41) | (SEQ ID NO: 48) | (SEQ ID NO: 63) |
| SASSSISYMH | SASYRYS | QQRSSYPYT |
| (SEQ ID NO: 41) | (SEQ ID NO: 48) | (SEQ ID NO: 64) |
| SASSSISYMH | SASYRYS | QHIREAYT |
| (SEQ ID NO: 41) | (SEQ ID NO: 48) | (SEQ ID NO: 65) |
| SASSSISYMH | DTSKLAS | QQYNSYPYT |
| (SEQ ID NO: 41) | (SEQ ID NO: 49) | (SEQ ID NO: 60) |
| SASSSISYMH | DTSKLAS | HQRSSYPLT |
| (SEQ ID NO: 41) | (SEQ ID NO: 49) | (SEQ ID NO: 61) |
| SASSSISYMH | DTSKLAS | LQYASYPYT |
| (SEQ ID NO: 41) | (SEQ ID NO: 49) | (SEQ ID NO: 62) |
| SASSSISYMH | DTSKLAS | FQGSHVPWT |
| (SEQ ID NO: 41) | (SEQ ID NO: 49) | (SEQ ID NO: 63) |
| SASSSISYMH | DTSKLAS | QQRSSYPYT |
| (SEQ ID NO: 41) | (SEQ ID NO: 49) | (SEQ ID NO: 64) |
| SASSSISYMH | DTSKLAS | QHIREAYT |
| (SEQ ID NO: 41) | (SEQ ID NO: 49) | (SEQ ID NO: 65) |
| SASSSISYMH | AASTLDS | QQYNSYPYT |
| (SEQ ID NO: 41) | (SEQ ID NO: 50) | (SEQ ID NO: 60) |
| SASSSISYMH | AASTLDS | HQRSSYPLT |
| (SEQ ID NO: 41) | (SEQ ID NO: 50) | (SEQ ID NO: 61) |
| SASSSISYMH | AASTLDS | LQYASYPYT |
| (SEQ ID NO: 41) | (SEQ ID NO: 50) | (SEQ ID NO: 62) |
| SASSSISYMH | AASTLDS | FQGSHVPWT |
| (SEQ ID NO: 41) | (SEQ ID NO: 50) | (SEQ ID NO: 63) |
| SASSSISYMH | AASTLDS | QQRSSYPYT |
| (SEQ ID NO: 41) | (SEQ ID NO: 50) | (SEQ ID NO: 64) |
| SASSSISYMH | AASTLDS | QHIREAYT |
| (SEQ ID NO: 41) | (SEQ ID NO: 50) | (SEQ ID NO: 65) |
| SASSSISYMH | KVSNRFS | QQYNSYPYT |
| (SEQ ID NO: 41) | (SEQ ID NO: 51) | (SEQ ID NO: 60) |
| SASSSISYMH | KVSNRFS | HQRSSYPLT |
| (SEQ ID NO: 41) | (SEQ ID NO: 51) | (SEQ ID NO: 61) |
| SASSSISYMH | KVSNRFS | LQYASYPYT |
| (SEQ ID NO: 41) | (SEQ ID NO: 51) | (SEQ ID NO: 62) |
| SASSSISYMH | KVSNRFS | FQGSHVPWT |
| (SEQ ID NO: 41) | (SEQ ID NO: 51) | (SEQ ID NO: 63) |
| SASSSISYMH | KVSNRFS | QQRSSYPYT |
| (SEQ ID NO: 41) | (SEQ ID NO: 51) | (SEQ ID NO: 64) |
| SASSSISYMH | KVSNRFS | QHIREAYT |
| (SEQ ID NO: 41) | (SEQ ID NO: 51) | (SEQ ID NO: 65) |
| SASSSISYMH | STSNLAS | QQYNSYPYT |
| (SEQ ID NO: 41) | (SEQ ID NO: 52) | (SEQ ID NO: 60) |
| SASSSISYMH | STSNLAS | HQRSSYPLT |
| (SEQ ID NO: 41) | (SEQ ID NO: 52) | (SEQ ID NO: 61) |
| SASSSISYMH | STSNLAS | LQYASYPYT |
| (SEQ ID NO: 41) | (SEQ ID NO: 52) | (SEQ ID NO: 62) |
| SASSSISYMH | STSNLAS | FQGSHVPWT |
| (SEQ ID NO: 41) | (SEQ ID NO: 52) | (SEQ ID NO: 63) |
| SASSSISYMH | STSNLAS | QQRSSYPYT |
| (SEQ ID NO: 41) | (SEQ ID NO: 52) | (SEQ ID NO: 64) |
| SASSSISYMH | STSNLAS | QHIREAYT |
| (SEQ ID NO: 41) | (SEQ ID NO: 52) | (SEQ ID NO: 65) |
| SASSSISYMH | LVSNLES | QQYNSYPYT |
| (SEQ ID NO: 41) | (SEQ ID NO: 53) | (SEQ ID NO: 60) |
| SASSSISYMH | LVSNLES | HQRSSYPLT |
| (SEQ ID NO: 41) | (SEQ ID NO: 53) | (SEQ ID NO: 61) |
| SASSSISYMH | LVSNLES | LQYASYPYT |
| (SEQ ID NO: 41) | (SEQ ID NO: 53) | (SEQ ID NO: 62) |
| SASSSISYMH | LVSNLES | FQGSHVPWT |
| (SEQ ID NO: 41) | (SEQ ID NO: 53) | (SEQ ID NO: 63) |
| SASSSISYMH | LVSNLES | QQRSSYPYT |
| (SEQ ID NO: 41) | (SEQ ID NO: 53) | (SEQ ID NO: 64) |
| SASSSISYMH | LVSNLES | QHIREAYT |
| (SEQ ID NO: 41) | (SEQ ID NO: 53) | (SEQ ID NO: 65) |
| RASQEISGYLS | SASYRYS | QQYNSYPYT |
| (SEQ ID NO: 42) | (SEQ ID NO: 48) | (SEQ ID NO: 60) |
| RASQEISGYLS | SASYRYS | HQRSSYPLT |
| (SEQ ID NO: 42) | (SEQ ID NO: 48) | (SEQ ID NO: 61) |
| RASQEISGYLS | SASYRYS | LQYASYPYT |
| (SEQ ID NO: 42) | (SEQ ID NO: 48) | (SEQ ID NO: 62) |
| RASQEISGYLS | SASYRYS | FQGSHVPWT |
| (SEQ ID NO: 42) | (SEQ ID NO: 48) | (SEQ ID NO: 63) |
| RASQEISGYLS | SASYRYS | QQRSSYPYT |
| (SEQ ID NO: 42) | (SEQ ID NO: 48) | (SEQ ID NO: 64) |
| RASQEISGYLS | SASYRYS | QHIREAYT |
| (SEQ ID NO: 42) | (SEQ ID NO: 48) | (SEQ ID NO: 65) |
| RASQEISGYLS | DTSKLAS | QQYNSYPYT |
| (SEQ ID NO: 42) | (SEQ ID NO: 49) | (SEQ ID NO: 60) |
| RASQEISGYLS | DTSKLAS | HQRSSYPLT |
| (SEQ ID NO: 42) | (SEQ ID NO: 49) | (SEQ ID NO: 61) |
| RASQEISGYLS | DTSKLAS | LQYASYPYT |
| (SEQ ID NO: 42) | (SEQ ID NO: 49) | (SEQ ID NO: 62) |
| RASQEISGYLS | DTSKLAS | FQGSHVPWT |
| (SEQ ID NO: 42) | (SEQ ID NO: 49) | (SEQ ID NO: 63) |
| RASQEISGYLS | DTSKLAS | QQRSSYPYT |
| (SEQ ID NO: 42) | (SEQ ID NO: 49) | (SEQ ID NO: 64) |
| RASQEISGYLS | DTSKLAS | QHIREAYT |
| (SEQ ID NO: 42) | (SEQ ID NO: 49) | (SEQ ID NO: 65) |
| RASQEISGYLS | AASTLDS | QQYNSYPYT |
| (SEQ ID NO: 42) | (SEQ ID NO: 50) | (SEQ ID NO: 60) |
| RASQEISGYLS | AASTLDS | HQRSSYPLT |
| (SEQ ID NO: 42) | (SEQ ID NO: 50) | (SEQ ID NO: 61) |
| RASQEISGYLS | AASTLDS | LQYASYPYT |
| (SEQ ID NO: 42) | (SEQ ID NO: 50) | (SEQ ID NO: 62) |
| RASQEISGYLS | AASTLDS | FQGSHVPWT |
| (SEQ ID NO: 42) | (SEQ ID NO: 50) | (SEQ ID NO: 63) |
| RASQEISGYLS | AASTLDS | QQRSSYPYT |
| (SEQ ID NO: 42) | (SEQ ID NO: 50) | (SEQ ID NO: 64) |
| RASQEISGYLS | AASTLDS | QHIREAYT |
| (SEQ ID NO: 42) | (SEQ ID NO: 50) | (SEQ ID NO: 65) |
| RASQEISGYLS | KVSNRFS | QQYNSYPYT |
| (SEQ ID NO: 42) | (SEQ ID NO: 51) | (SEQ ID NO: 60) |
| RASQEISGYLS | KVSNRFS | HQRSSYPLT |
| (SEQ ID NO: 42) | (SEQ ID NO: 51) | (SEQ ID NO: 61) |
| RASQEISGYLS | KVSNRFS | LQYASYPYT |
| (SEQ ID NO: 42) | (SEQ ID NO: 51) | (SEQ ID NO: 62) |
| RASQEISGYLS | KVSNRFS | FQGSHVPWT |
| (SEQ ID NO: 42) | (SEQ ID NO: 51) | (SEQ ID NO: 63) |
| RASQEISGYLS | KVSNRFS | QQRSSYPYT |
| (SEQ ID NO: 42) | (SEQ ID NO: 51) | (SEQ ID NO: 64) |
| RASQEISGYLS | KVSNRFS | QHIREAYT |
| (SEQ ID NO: 42) | (SEQ ID NO: 51) | (SEQ ID NO: 65) |
| RASQEISGYLS | STSNLAS | QQYNSYPYT |
| (SEQ ID NO: 42) | (SEQ ID NO: 52) | (SEQ ID NO: 60) |
| RASQEISGYLS | STSNLAS | HQRSSYPLT |
| (SEQ ID NO: 42) | (SEQ ID NO: 52) | (SEQ ID NO: 61) |
| RASQEISGYLS | STSNLAS | LQYASYPYT |
| (SEQ ID NO: 42) | (SEQ ID NO: 52) | (SEQ ID NO: 62) |
| RASQEISGYLS | STSNLAS | FQGSHVPWT |
| (SEQ ID NO: 42) | (SEQ ID NO: 52) | (SEQ ID NO: 63) |
| RASQEISGYLS | STSNLAS | QQRSSYPYT |
| (SEQ ID NO: 42) | (SEQ ID NO: 52) | (SEQ ID NO: 64) |
| RASQEISGYLS | STSNLAS | QHIREAYT |
| (SEQ ID NO: 42) | (SEQ ID NO: 52) | (SEQ ID NO: 65) |
| RASQEISGYLS | LVSNLES | QQYNSYPYT |
| (SEQ ID NO: 42) | (SEQ ID NO: 53) | (SEQ ID NO: 60) |
| RASQEISGYLS | LVSNLES | HQRSSYPLT |
| (SEQ ID NO: 42) | (SEQ ID NO: 53) | (SEQ ID NO: 61) |
| RASQEISGYLS | LVSNLES | LQYASYPYT |
| (SEQ ID NO: 42) | (SEQ ID NO: 53) | (SEQ ID NO: 62) |
| RASQEISGYLS | LVSNLES | FQGSHVPWT |
| (SEQ ID NO: 42) | (SEQ ID NO: 53) | (SEQ ID NO: 63) |
| RASQEISGYLS | LVSNLES | QQRSSYPYT |
| (SEQ ID NO: 42) | (SEQ ID NO: 53) | (SEQ ID NO: 64) |
| RASQEISGYLS | LVSNLES | QHIREAYT |
| (SEQ ID NO: 42) | (SEQ ID NO: 53) | (SEQ ID NO: 65) |
| RSSQSIVHSNGNTYLE | SASYRYS | QQYNSYPYT |
| (SEQ ID NO: 43) | (SEQ ID NO: 48) | (SEQ ID NO: 60) |
| RSSQSIVHSNGNTYLE | SASYRYS | HQRSSYPLT |
| (SEQ ID NO: 43) | (SEQ ID NO: 48) | (SEQ ID NO: 61) |
| RSSQSIVHSNGNTYLE | SASYRYS | LQYASYPYT |
| (SEQ ID NO: 43) | (SEQ ID NO: 48) | (SEQ ID NO: 62) |
| RSSQSIVHSNGNTYLE | SASYRYS | FQGSHVPWT |
| (SEQ ID NO: 43) | (SEQ ID NO: 48) | (SEQ ID NO: 63) |
| RSSQSIVHSNGNTYLE | SASYRYS | QQRSSYPYT |
| (SEQ ID NO: 43) | (SEQ ID NO: 48) | (SEQ ID NO: 64) |
| RSSQSIVHSNGNTYLE | SASYRYS | QHIREAYT |
| (SEQ ID NO: 43) | (SEQ ID NO: 48) | (SEQ ID NO: 65) |
| RSSQSIVHSNGNTYLE | DTSKLAS | QQYNSYPYT |
| (SEQ ID NO: 43) | (SEQ ID NO: 49) | (SEQ ID NO: 60) |
| RSSQSIVHSNGNTYLE | DTSKLAS | HQRSSYPLT |
| (SEQ ID NO: 43) | (SEQ ID NO: 49) | (SEQ ID NO: 61) |
| RSSQSIVHSNGNTYLE | DTSKLAS | LQYASYPYT |
| (SEQ ID NO: 43) | (SEQ ID NO: 49) | (SEQ ID NO: 62) |
| RSSQSIVHSNGNTYLE | DTSKLAS | FQGSHVPWT |
| (SEQ ID NO: 43) | (SEQ ID NO: 49) | (SEQ ID NO: 63) |
| RSSQSIVHSNGNTYLE | DTSKLAS | QQRSSYPYT |
| (SEQ ID NO: 43) | (SEQ ID NO: 49) | (SEQ ID NO: 64) |
| RSSQSIVHSNGNTYLE | DTSKLAS | QHIREAYT |
| (SEQ ID NO: 43) | (SEQ ID NO: 49) | (SEQ ID NO: 65) |
| RSSQSIVHSNGNTYLE | AASTLDS | QQYNSYPYT |
| (SEQ ID NO: 43) | (SEQ ID NO: 50) | (SEQ ID NO: 60) |
| RSSQSIVHSNGNTYLE | AASTLDS | HQRSSYPLT |
| (SEQ ID NO: 43) | (SEQ ID NO: 50) | (SEQ ID NO: 61) |
| RSSQSIVHSNGNTYLE | AASTLDS | LQYASYPYT |
| (SEQ ID NO: 43) | (SEQ ID NO: 50) | (SEQ ID NO: 62) |
| RSSQSIVHSNGNTYLE | AASTLDS | FQGSHVPWT |
| (SEQ ID NO: 43) | (SEQ ID NO: 50) | (SEQ ID NO: 63) |
| RSSQSIVHSNGNTYLE | AASTLDS | QQRSSYPYT |
| (SEQ ID NO: 43) | (SEQ ID NO: 50) | (SEQ ID NO: 64) |
| RSSQSIVHSNGNTYLE | AASTLDS | QHIREAYT |
| (SEQ ID NO: 43) | (SEQ ID NO: 50) | (SEQ ID NO: 65) |
| RSSQSIVHSNGNTYLE | KVSNRFS | QQYNSYPYT |
| (SEQ ID NO: 43) | (SEQ ID NO: 51) | (SEQ ID NO: 60) |
| RSSQSIVHSNGNTYLE | KVSNRFS | HQRSSYPLT |
| (SEQ ID NO: 43) | (SEQ ID NO: 51) | (SEQ ID NO: 61) |
| RSSQSIVHSNGNTYLE | KVSNRFS | LQYASYPYT |
| (SEQ ID NO: 43) | (SEQ ID NO: 51) | (SEQ ID NO: 62) |
| RSSQSIVHSNGNTYLE | KVSNRFS | FQGSHVPWT |
| (SEQ ID NO: 43) | (SEQ ID NO: 51) | (SEQ ID NO: 63) |
| RSSQSIVHSNGNTYLE | KVSNRFS | QQRSSYPYT |
| (SEQ ID NO: 43) | (SEQ ID NO: 51) | (SEQ ID NO: 64) |
| RSSQSIVHSNGNTYLE | KVSNRFS | QHIREAYT |
| (SEQ ID NO: 43) | (SEQ ID NO: 51) | (SEQ ID NO: 65) |
| RSSQSIVHSNGNTYLE | STSNLAS | QQYNSYPYT |
| (SEQ ID NO: 43) | (SEQ ID NO: 52) | (SEQ ID NO: 60) |
| RSSQSIVHSNGNTYLE | STSNLAS | HQRSSYPLT |
| (SEQ ID NO: 43) | (SEQ ID NO: 52) | (SEQ ID NO: 61) |
| RSSQSIVHSNGNTYLE | STSNLAS | LQYASYPYT |
| (SEQ ID NO: 43) | (SEQ ID NO: 52) | (SEQ ID NO: 62) |
| RSSQSIVHSNGNTYLE | STSNLAS | FQGSHVPWT |
| (SEQ ID NO: 43) | (SEQ ID NO: 52) | (SEQ ID NO: 63) |
| RSSQSIVHSNGNTYLE | STSNLAS | QQRSSYPYT |
| (SEQ ID NO: 43) | (SEQ ID NO: 52) | (SEQ ID NO: 64) |
| RSSQSIVHSNGNTYLE | STSNLAS | QHIREAYT |
| (SEQ ID NO: 43) | (SEQ ID NO: 52) | (SEQ ID NO: 65) |
| RSSQSIVHSNGNTYLE | LVSNLES | QQYNSYPYT |
| (SEQ ID NO: 43) | (SEQ ID NO: 53) | (SEQ ID NO: 60) |
| RSSQSIVHSNGNTYLE | LVSNLES | HQRSSYPLT |
| (SEQ ID NO: 43) | (SEQ ID NO: 53) | (SEQ ID NO: 61) |
| RSSQSIVHSNGNTYLE | LVSNLES | LQYASYPYT |
| (SEQ ID NO: 43) | (SEQ ID NO: 53) | (SEQ ID NO: 62) |
| RSSQSIVHSNGNTYLE | LVSNLES | FQGSHVPWT |
| (SEQ ID NO: 43) | (SEQ ID NO: 53) | (SEQ ID NO: 63) |
| RSSQSIVHSNGNTYLE | LVSNLES | QQRSSYPYT |
| (SEQ ID NO: 43) | (SEQ ID NO: 53) | (SEQ ID NO: 64) |
| RSSQSIVHSNGNTYLE | LVSNLES | QHIREAYT |
| (SEQ ID NO: 43) | (SEQ ID NO: 53) | (SEQ ID NO: 65) |
| SASSSVSYMH | SASYRYS | QQYNSYPYT |
| (SEQ ID NO: 44) | (SEQ ID NO: 48) | (SEQ ID NO: 60) |
| SASSSVSYMH | SASYRYS | HQRSSYPLT |
| (SEQ ID NO: 44) | (SEQ ID NO: 48) | (SEQ ID NO: 61) |
| SASSSVSYMH | SASYRYS | LQYASYPYT |
| (SEQ ID NO: 44) | (SEQ ID NO: 48) | (SEQ ID NO: 62) |
| SASSSVSYMH | SASYRYS | FQGSHVPWT |
| (SEQ ID NO: 44) | (SEQ ID NO: 48) | (SEQ ID NO: 63) |
| SASSSVSYMH | SASYRYS | QQRSSYPYT |
| (SEQ ID NO: 44) | (SEQ ID NO: 48) | (SEQ ID NO: 64) |
| SASSSVSYMH | SASYRYS | QHIREAYT |
| (SEQ ID NO: 44) | (SEQ ID NO: 48) | (SEQ ID NO: 65) |
| SASSSVSYMH | DTSKLAS | QQYNSYPYT |
| (SEQ ID NO: 44) | (SEQ ID NO: 49) | (SEQ ID NO: 60) |
| SASSSVSYMH | DTSKLAS | HQRSSYPLT |
| (SEQ ID NO: 44) | (SEQ ID NO: 49) | (SEQ ID NO: 61) |
| SASSSVSYMH | DTSKLAS | LQYASYPYT |
| (SEQ ID NO: 44) | (SEQ ID NO: 49) | (SEQ ID NO: 62) |
| SASSSVSYMH | DTSKLAS | FQGSHVPWT |
| (SEQ ID NO: 44) | (SEQ ID NO: 49) | (SEQ ID NO: 63) |
| SASSSVSYMH | DTSKLAS | QQRSSYPYT |
| (SEQ ID NO: 44) | (SEQ ID NO: 49) | (SEQ ID NO: 64) |
| SASSSVSYMH | DTSKLAS | QHIREAYT |
| (SEQ ID NO: 44) | (SEQ ID NO: 49) | (SEQ ID NO: 65) |
| SASSSVSYMH | AASTLDS | QQYNSYPYT |
| (SEQ ID NO: 44) | (SEQ ID NO: 50) | (SEQ ID NO: 60) |
| SASSSVSYMH | AASTLDS | HQRSSYPLT |
| (SEQ ID NO: 44) | (SEQ ID NO: 50) | (SEQ ID NO: 61) |
| SASSSVSYMH | AASTLDS | LQYASYPYT |
| (SEQ ID NO: 44) | (SEQ ID NO: 50) | (SEQ ID NO: 62) |
| SASSSVSYMH | AASTLDS | FQGSHVPWT |
| (SEQ ID NO: 44) | (SEQ ID NO: 50) | (SEQ ID NO: 63) |
| SASSSVSYMH | AASTLDS | QQRSSYPYT |
| (SEQ ID NO: 44) | (SEQ ID NO: 50) | (SEQ ID NO: 64) |
| SASSSVSYMH | AASTLDS | QHIREAYT |
| (SEQ ID NO: 44) | (SEQ ID NO: 50) | (SEQ ID NO: 65) |
| SASSSVSYMH | KVSNRFS | QQYNSYPYT |
| (SEQ ID NO: 44) | (SEQ ID NO: 51) | (SEQ ID NO: 60) |
| SASSSVSYMH | KVSNRFS | HQRSSYPLT |
| (SEQ ID NO: 44) | (SEQ ID NO: 51) | (SEQ ID NO: 61) |
| SASSSVSYMH | KVSNRFS | LQYASYPYT |
| (SEQ ID NO: 44) | (SEQ ID NO: 51) | (SEQ ID NO: 62) |
| SASSSVSYMH | KVSNRFS | FQGSHVPWT |
| (SEQ ID NO: 44) | (SEQ ID NO: 51) | (SEQ ID NO: 63) |
| SASSSVSYMH | KVSNRFS | QQRSSYPYT |
| (SEQ ID NO: 44) | (SEQ ID NO: 51) | (SEQ ID NO: 64) |
| SASSSVSYMH | KVSNRFS | QHIREAYT |
| (SEQ ID NO: 44) | (SEQ ID NO: 51) | (SEQ ID NO: 65) |
| SASSSVSYMH | STSNLAS | QQYNSYPYT |
| (SEQ ID NO: 44) | (SEQ ID NO: 52) | (SEQ ID NO: 60) |
| SASSSVSYMH | STSNLAS | HQRSSYPLT |
| (SEQ ID NO: 44) | (SEQ ID NO: 52) | (SEQ ID NO: 61) |
| SASSSVSYMH | STSNLAS | LQYASYPYT |
| (SEQ ID NO: 44) | (SEQ ID NO: 52) | (SEQ ID NO: 62) |
| SASSSVSYMH | STSNLAS | FQGSHVPWT |
| (SEQ ID NO: 44) | (SEQ ID NO: 52) | (SEQ ID NO: 63) |
| SASSSVSYMH | STSNLAS | QQRSSYPYT |
| (SEQ ID NO: 44) | (SEQ ID NO: 52) | (SEQ ID NO: 64) |
| SASSSVSYMH | STSNLAS | QHIREAYT |
| (SEQ ID NO: 44) | (SEQ ID NO: 52) | (SEQ ID NO: 65) |
| SASSSVSYMH | LVSNLES | QQYNSYPYT |
| (SEQ ID NO: 44) | (SEQ ID NO: 53) | (SEQ ID NO: 60) |
| SASSSVSYMH | LVSNLES | HQRSSYPLT |
| (SEQ ID NO: 44) | (SEQ ID NO: 53) | (SEQ ID NO: 61) |
| SASSSVSYMH | LVSNLES | LQYASYPYT |
| (SEQ ID NO: 44) | (SEQ ID NO: 53) | (SEQ ID NO: 62) |
| SASSSVSYMH | LVSNLES | FQGSHVPWT |
| (SEQ ID NO: 44) | (SEQ ID NO: 53) | (SEQ ID NO: 63) |
| SASSSVSYMH | LVSNLES | QQRSSYPYT |
| (SEQ ID NO: 44) | (SEQ ID NO: 53) | (SEQ ID NO: 64) |
| SASSSVSYMH | LVSNLES | QHIREAYT |
| (SEQ ID NO: 44) | (SEQ ID NO: 53) | (SEQ ID NO: 65) |
| KSVSTSGYSYMH | SASYRYS | QQYNSYPYT |
| (SEQ ID NO: 45) | (SEQ ID NO: 48) | (SEQ ID NO: 60) |
| KSVSTSGYSYMH | SASYRYS | HQRSSYPLT |
| (SEQ ID NO: 45) | (SEQ ID NO: 48) | (SEQ ID NO: 61) |
| KSVSTSGYSYMH | SASYRYS | LQYASYPYT |
| (SEQ ID NO: 45) | (SEQ ID NO: 48) | (SEQ ID NO: 62) |
| KSVSTSGYSYMH | SASYRYS | FQGSHVPWT |
| (SEQ ID NO: 45) | (SEQ ID NO: 48) | (SEQ ID NO: 63) |
| KSVSTSGYSYMH | SASYRYS | QQRSSYPYT |
| (SEQ ID NO: 45) | (SEQ ID NO: 48) | (SEQ ID NO: 64) |
| KSVSTSGYSYMH | SASYRYS | QHIREAYT |
| (SEQ ID NO: 45) | (SEQ ID NO: 48) | (SEQ ID NO: 65) |
| KSVSTSGYSYMH | DTSKLAS | QQYNSYPYT |
| (SEQ ID NO: 45) | (SEQ ID NO: 49) | (SEQ ID NO: 60) |
| KSVSTSGYSYMH | DTSKLAS | HQRSSYPLT |
| (SEQ ID NO: 45) | (SEQ ID NO: 49) | (SEQ ID NO: 61) |
| KSVSTSGYSYMH | DTSKLAS | LQYASYPYT |
| (SEQ ID NO: 45) | (SEQ ID NO: 49) | (SEQ ID NO: 62) |
| KSVSTSGYSYMH | DTSKLAS | FQGSHVPWT |
| (SEQ ID NO: 45) | (SEQ ID NO: 49) | (SEQ ID NO: 63) |
| KSVSTSGYSYMH | DTSKLAS | QQRSSYPYT |
| (SEQ ID NO: 45) | (SEQ ID NO: 49) | (SEQ ID NO: 64) |
| KSVSTSGYSYMH | DTSKLAS | QHIREAYT |
| (SEQ ID NO: 45) | (SEQ ID NO: 49) | (SEQ ID NO: 65) |
| KSVSTSGYSYMH | AASTLDS | QQYNSYPYT |
| (SEQ ID NO: 45) | (SEQ ID NO: 50) | (SEQ ID NO: 60) |
| KSVSTSGYSYMH | AASTLDS | HQRSSYPLT |
| (SEQ ID NO: 45) | (SEQ ID NO: 50) | (SEQ ID NO: 61) |
| KSVSTSGYSYMH | AASTLDS | LQYASYPYT |
| (SEQ ID NO: 45) | (SEQ ID NO: 50) | (SEQ ID NO: 62) |
| KSVSTSGYSYMH | AASTLDS | FQGSHVPWT |
| (SEQ ID NO: 45) | (SEQ ID NO: 50) | (SEQ ID NO: 63) |
| KSVSTSGYSYMH | AASTLDS | QQRSSYPYT |
| (SEQ ID NO: 45) | (SEQ ID NO: 50) | (SEQ ID NO: 64) |
| KSVSTSGYSYMH | AASTLDS | QHIREAYT |
| (SEQ ID NO: 45) | (SEQ ID NO: 50) | (SEQ ID NO: 65) |
| KSVSTSGYSYMH | KVSNRFS | QQYNSYPYT |
| (SEQ ID NO: 45) | (SEQ ID NO: 51) | (SEQ ID NO: 60) |
| KSVSTSGYSYMH | KVSNRFS | HQRSSYPLT |
| (SEQ ID NO: 45) | (SEQ ID NO: 51) | (SEQ ID NO: 61) |
| KSVSTSGYSYMH | KVSNRFS | LQYASYPYT |
| (SEQ ID NO: 45) | (SEQ ID NO: 51) | (SEQ ID NO: 62) |
| KSVSTSGYSYMH | KVSNRFS | FQGSHVPWT |
| (SEQ ID NO: 45) | (SEQ ID NO: 51) | (SEQ ID NO: 63) |
| KSVSTSGYSYMH | KVSNRFS | QQRSSYPYT |
| (SEQ ID NO: 45) | (SEQ ID NO: 51) | (SEQ ID NO: 64) |
| KSVSTSGYSYMH | KVSNRFS | QHIREAYT |
| (SEQ ID NO: 45) | (SEQ ID NO: 51) | (SEQ ID NO: 65) |
| KSVSTSGYSYMH | STSNLAS | QQYNSYPYT |
| (SEQ ID NO: 45) | (SEQ ID NO: 52) | (SEQ ID NO: 60) |
| KSVSTSGYSYMH | STSNLAS | HQRSSYPLT |
| (SEQ ID NO: 45) | (SEQ ID NO: 52) | (SEQ ID NO: 61) |
| KSVSTSGYSYMH | STSNLAS | LQYASYPYT |
| (SEQ ID NO: 45) | (SEQ ID NO: 52) | (SEQ ID NO: 62) |
| KSVSTSGYSYMH | STSNLAS | FQGSHVPWT |
| (SEQ ID NO: 45) | (SEQ ID NO: 52) | (SEQ ID NO: 63) |
| KSVSTSGYSYMH | STSNLAS | QQRSSYPYT |
| (SEQ ID NO: 45) | (SEQ ID NO: 52) | (SEQ ID NO: 64) |
| KSVSTSGYSYMH | STSNLAS | QHIREAYT |
| (SEQ ID NO: 45) | (SEQ ID NO: 52) | (SEQ ID NO: 65) |
| KSVSTSGYSYMH | LVSNLES | QQYNSYPYT |
| (SEQ ID NO: 45) | (SEQ ID NO: 53) | (SEQ ID NO: 60) |
| KSVSTSGYSYMH | LVSNLES | HQRSSYPLT |
| (SEQ ID NO: 45) | (SEQ ID NO: 53) | (SEQ ID NO: 61) |
| KSVSTSGYSYMH | LVSNLES | LQYASYPYT |
| (SEQ ID NO: 45) | (SEQ ID NO: 53) | (SEQ ID NO: 62) |
| KSVSTSGYSYMH | LVSNLES | FQGSHVPWT |
| (SEQ ID NO: 45) | (SEQ ID NO: 53) | (SEQ ID NO: 63) |
| KSVSTSGYSYMH | LVSNLES | QQRSSYPYT |
| (SEQ ID NO: 45) | (SEQ ID NO: 53) | (SEQ ID NO: 64) |
| KSVSTSGYSYMH | LVSNLES | QHIREAYT |
| (SEQ ID NO: 45) | (SEQ ID NO: 53) | (SEQ ID NO: 65) |

1. A chimeric antigen receptor (CAR), comprising:
(a) an extracellular region comprising a binding domain that specifically binds to at least a portion of B7-H3;
(b) a transmembrane region; and
(c) an intracellular region comprising an effector domain or a portion or variant thereof and a costimulatory domain or a portion or variant thereof.

2. The CAR of claim 1, wherein the binding domain includes at least a V_{L} chain of an antibody which binds to B7-H3 or a portion or variant thereof and a V_{H} chain of an antibody which binds to B7-H3 or a portion or variant thereof.

3. (canceled)

4. (canceled)

5. The CAR of claim 2, wherein the V_{L} chain comprises an amino acid sequence having at least 75% identity to the amino acid sequence shown in SEQ ID NO: 8, SEQ ID NO: 10, SEQ ID NO: 12, SEQ ID NO: 14, SEQ ID NO: 16, or SEQ ID NO: 18.

6. (canceled)

7. The CAR of claim 4, wherein the V_{H} chain comprises an amino acid sequence having at least 75% identity to the amino acid sequence shown in SEQ ID NO: 7, SEQ ID NO: 9, SEQ ID NO: 11, SEQ ID NO: 13, SEQ ID NO: 15, or SEQ ID NO: 17.

8. (canceled)

9. The CAR of claim 6, wherein the binding domain is an scFv.

10. The CAR of claim 9, wherein the extracellular region further comprises a linker or a portion or variant thereof.

11. The CAR of claim 10, wherein the linker is a glycine-serine linker.

12. (canceled)

13. (canceled)

14. The CAR of claim 11, wherein the extracellular region further comprises a leader or a portion or variant thereof.

15. (canceled)

16. (canceled)

17. The CAR of claim 14, wherein the transmembrane region comprises or is a combination of (i) a CD8α hinge or a portion or variant thereof and (ii) CD8α transmembrane region or a portion or variant thereof.

18. (canceled)

19. The CAR of claim 17, wherein the effector domain or portion or variant thereof is CD3ζ or a portion or variant thereof.

20. (canceled)

21. The CAR of claim 19, wherein the costimulatory domain or portion or variant thereof is a CD28 costimulatory domain or a portion or variant thereof, a 4-1BB costimulatory domain or a portion or variant thereof, or a combination thereof.

22. (canceled)

23. (canceled)

24. The CAR claim 21, wherein the costimulatory domain comprises a CD28 costimulatory domain or a portion or variant thereof and the effector domain comprises CD3ζ or a portion or variant thereof.

25. (canceled)

26. The CAR of claim 21, wherein the costimulatory domain comprises a 4-1BB costimulatory domain or a portion or variant thereof and the effector domain comprises CD3ζ or a portion or variant thereof.

27. (canceled)

28. The CAR of claim 21, wherein the costimulatory domain comprises a CD28 costimulatory domain or a portion or variant thereof and a 4-1BB costimulatory domain or a portion or variant thereof, and the effector domain comprises CD3ζ or a portion or variant thereof.

29. (canceled)

30. The CAR claim 28, wherein the binding domain is chimeric, human, or humanized.

31. (canceled)

32. An expression vector, comprising an isolated polynucleotide encoding the CAR of claim 28 operably linked to an expression control sequence.

33. (canceled)

34. The expression vector of claim 32, further comprising an isolated polynucleotide encoding a self-cleaving peptide.

35. The expression vector of claim 34, wherein the self-cleaving peptide is a 2A self-cleaving peptide or a P2A peptide.

36. (canceled)

37. The expression vector of claim 36, further comprising an isolated polynucleotide encoding a transduction marker polypeptide.

38. The expression vector of claim 37, wherein the transduction marker polypeptide is a truncated form of epidermal growth factor receptor (EGFRt) or a portion or variant thereof or GFP or a portion or variant thereof.

39.-63. (canceled)


SEQUENCE LISTING (121 sequences)

SEQ ID NO: 1 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 2 - protein, 41 aa, Homo sapiens
RSKRSRLLHSDYMNMTPRRPGPTRKHYQPYAPPRDFAAYRS

SEQ ID NO: 3 - protein, 42 aa, Homo sapiens
KRGRKKLLYIFKQPFMRPVQTTQEEDGCSCRFPEEEEGGCEL

SEQ ID NO: 4 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 22 aa, Homo sapiens
GSGATNFSLLKQAGDVEENPGP

SEQ ID NO: 6 - protein, 335 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 7 - protein, 134 aa, Artificial Sequence
  1G5 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(117): FR3
  MISC_FEATURE (118)..(123): CDR3
  MISC_FEATURE (124)..(134): FR4
MGWSYIILFLVATATGVHSQVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARGGYFDYWGQGTTLTVSS

SEQ ID NO: 8 - protein, 128 aa, Artificial Sequence
  1G5 mAb VL chain
  MISC_FEATURE (1)..(20): Leader sequence
  MISC_FEATURE (21)..(43): FR1
  MISC_FEATURE (44)..(54): CDR1
  MISC_FEATURE (55)..(69): FR2
  MISC_FEATURE (70)..(76): CDR2
  MISC_FEATURE (77)..(108): FR3
  MISC_FEATURE (109)..(117): CDR3
  MISC_FEATURE (118)..(128): FR4
MKTQTQVFVYMLLWLSGVDGDIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIKR

SEQ ID NO: 9 - protein, 135 aa, Artificial Sequence
  23B2 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(115): FR3
  MISC_FEATURE (116)..(124): CDR3
  MISC_FEATURE (125)..(135): FR4
MGWSCIMLFLVATATGVHSQVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARESRQLDYWGQGTTLTVSS

SEQ ID NO: 10 - protein, 128 aa, Artificial Sequence
  23B2 mAb VL chain
  MISC_FEATURE (1)..(22): Leader sequence
  MISC_FEATURE (23)..(45): FR1
  MISC_FEATURE (46)..(55): CDR1
  MISC_FEATURE (56)..(70): FR2
  MISC_FEATURE (71)..(77): CDR2
  MISC_FEATURE (78)..(109): FR3
  MISC_FEATURE (110)..(118): CDR3
  MISC_FEATURE (119)..(128): FR4
MDLQVQIFSFLLISASVILSRGQIVLTQSPAIMSASPGEKVTMTCSASSSISYMHWYQQKPGTSPKRWIYDTSKLASGVPARFSGSGSGTSYSLTISSMEAEDAATYYCHQRSSYPLTFGAGTKLELK

SEQ ID NO: 11 - protein, 134 aa, Artificial Sequence
  15F9 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(117): FR3
  MISC_FEATURE (118)..(123): CDR3
  MISC_FEATURE (124)..(134): FR4
MGWSCIMFFLVATATGVHSQVQLQQPGAELVKPGASVKLSCKASGYTFTNYWMHWVKQRPGQGLEWIGMIHPNSGSTNYNEKFKSKATLTVDKSSSTAYMQLSSLTSEDSAVYYCARRGDGDYWGQGTTLTVSS

SEQ ID NO: 12 - protein, 121 aa, Artificial Sequence
  15F9 mAb VL chain
  MISC_FEATURE (1)..(15): Leader sequence
  MISC_FEATURE (16)..(38): FR1
  MISC_FEATURE (39)..(49): CDR1
  MISC_FEATURE (50)..(64): FR2
  MISC_FEATURE (65)..(71): CDR2
  MISC_FEATURE (72)..(103): FR3
  MISC_FEATURE (104)..(112): CDR3
  MISC_FEATURE (113)..(121): FR4
MVLISLLFWFPGTRCDIQMTRHPSSLSASMGERVSLTCRASQEISGYLSWLQQKPDGTIKRLIYAASTLDSGVPKRFSGSRSGSDYSLTISSLESEDFADYYCLQYASYPYTFGGGTKLEI

SEQ ID NO: 13 - protein, 139 aa, Artificial Sequence
  8B12 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(115): FR3
  MISC_FEATURE (116)..(127): CDR3
  MISC_FEATURE (128)..(139): FR4
MEWSWVILILVAAATGVHSQVQLQQPGAELVKPGASVKMSCKASGYTFTSYWITWVKQRPGQGLEWIGDIYPGSGSTNYNEKFKSKATLTVDTSSSTAYMQLSSLTSEDSAVYYCARGGTRFSPFAYWGQGTLVTVSAA

SEQ ID NO: 14 - protein, 135 aa, Artificial Sequence
  8B12 mAb VL chain
  MISC_FEATURE (1)..(19): Leader sequence
  MISC_FEATURE (20)..(42): FR1
  MISC_FEATURE (43)..(58): CDR1
  MISC_FEATURE (59)..(73): FR2
  MISC_FEATURE (74)..(80): CDR2
  MISC_FEATURE (81)..(112): FR3
  MISC_FEATURE (113)..(121): CDR3
  MISC_FEATURE (122)..(135): FR4
MKMPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKRADA

SEQ ID NO: 15 - protein, 138 aa, Artificial Sequence
  12B4 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(117): FR3
  MISC_FEATURE (118)..(128): CDR3
  MISC_FEATURE (129)..(138): FR4
MGWSCIMFFLVATATGVHSQVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARYYYGSSYAMDYWGQGTSVTVS

SEQ ID NO: 16 - protein, 131 aa, Artificial Sequence
  12B4 mAb VL chain
  MISC_FEATURE (1)..(22): Leader sequence
  MISC_FEATURE (23)..(45): FR1
  MISC_FEATURE (46)..(55): CDR1
  MISC_FEATURE (56)..(70): FR2
  MISC_FEATURE (71)..(77): CDR2
  MISC_FEATURE (78)..(109): FR3
  MISC_FEATURE (110)..(118): CDR3
  MISC_FEATURE (119)..(131): FR4
MDFQVQIFSFLLISASVIMSRGQIVLTQSPAIMSASPGEKVTITCSASSSVSYMHWFQQKPGTSPKLWIYSTSNLASGVPARFSGSGSGTSYSLTISRMEAEDAATYYCQQRSSYPYTFGGGTKLEIKRAD

SEQ ID NO: 17 - protein, 133 aa, Artificial Sequence
  24D12 mAb VH chain
  MISC_FEATURE (1)..(19): Leader sequence
  MISC_FEATURE (20)..(44): FR1
  MISC_FEATURE (45)..(54): CDR1
  MISC_FEATURE (55)..(68): FR2
  MISC_FEATURE (69)..(85): CDR2
  MISC_FEATURE (86)..(117): FR3
  MISC_FEATURE (118)..(123): CDR3
  MISC_FEATURE (124)..(133): FR4
MGWSCIMFFLVATATGVHSQVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCANYYGTNVWGTGTTVTVS

SEQ ID NO: 18 - protein, 129 aa, Artificial Sequence
  24D12 mAb VL chain
  MISC_FEATURE (1)..(20): Leader sequence
  MISC_FEATURE (21)..(46): FR1
  MISC_FEATURE (47)..(58): CDR1
  MISC_FEATURE (59)..(73): FR2
  MISC_FEATURE (74)..(80): CDR2
  MISC_FEATURE (81)..(112): FR3
  MISC_FEATURE (113)..(120): CDR3
  MISC_FEATURE (121)..(129): FR4
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIREAYTFGRGDQSWK

SEQ ID NO: 19 - DNA, 207 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 20 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 21 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 22 - DNA, 342 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 23 - DNA, 66 bp, Homo sapiens
ggaagcggagctactaacttcagcctgctgaagcaggctggagacgtggaggagaaccctggacct

SEQ ID NO: 24 - DNA, 1005 bp, Homo sapiens
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 25 - DNA, 402 bp, Artificial Sequence
  1G5 mAb VH chain
atgggatggagctatatcatcctctttttggtagcaacagctacaggtgtccactcccaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaaggggggggtactttgactactggggccaaggcaccactctcacagtctcctca

SEQ ID NO: 26 - DNA, 384 bp, Artificial Sequence
  1G5 mAb VL chain
atgaagacacagactcaggtctttgtatacatgttgctgtggttgtctggtgttgatggagacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaacgg

SEQ ID NO: 27 - DNA, 405 bp, Artificial Sequence
  23B2 mAb VL chain
atgggatggagctgtatcatgctctttttggtagcaacagctacaggtgtccactcccaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagagagagtagacagctcgactactggggccaaggcaccactctcacagtctcctca

SEQ ID NO: 28 - DNA, 384 bp, Artificial Sequence
  23B2 mAb VL chain
atggatttacaagtgcagattttcagcttcctgctaatcagtgcctcagtcatactgtccagaggacaaattgttctcacccagtctccagcaatcatgtctgcatctccaggggagaaggtcaccatgacctgcagtgccagctcaagtataagttacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacccgctcacgttcggtgctgggaccaagctggagctgaaa

SEQ ID NO: 29 - DNA, 403 bp, Artificial Sequence
  15F9 mAb VH chain
atgggatggagctgtatcatgttctttttggtagcaacagctacaggtgtccactcccaggtccaactgcagcagcctggggctgagctggtaaagcctggggcttcagtgaagttgtcctgcaaggcttctggctacactttcaccaactactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaaggcgaggggacggggactactggggccaaggcaccactctcacagtctcctcag

SEQ ID NO: 30 - DNA, 363 bp, Artificial Sequence
  15F9 mAb VL chain
atggttcttatttccttgctgttctggtttccaggtaccagatgtgacatccagatgacccggcatccttcttctttatctgcctctatgggagaaagagtcagtctcacttgtcgggcaagtcaggaaattagtggttacttaagctggcttcagcagaaaccagatggaactattaaacgcctgatttacgccgcatccactttagattctggtgtcccaaaaaggttcagtggcagtaggtctgggtcagattattctctcaccatcagcagccttgagtctgaagattttgcagactattactgtctacaatatgctagttatccgtacacgttcggaggggggaccaagctggaaata

SEQ ID NO: 31 - DNA, 419 bp, Artificial Sequence
  8B12 mAb VH chain
atggaatggagctgggtcatcctcattttggtagcagcagctacaggtgtccactcccaggtccaactgcagcagcctggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtagtactaactacaatgagaagttcaagagcaaggccactctgactgtagacacatcctccagcacagcctacatgcagctcagcagcctgacatctgaggactctgcggtctattactgtgcaagagggggtactagattcagcccctttgcttactggggccaagggactctggtcactgtctctgcagctac

SEQ ID NO: 32 - DNA, 405 bp, Artificial Sequence
  8B12 mAb VL chain
atgaagatgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaacgggctgatgct

SEQ ID NO: 33 - DNA, 416 bp, Artificial Sequence
  12B4 mAb VH chain
atgggatggagctgtatcatgttctttttggtagcaacagctacaggtgtccactcccaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagatattactacggtagtagctatgctatggactactggggtcaaggaacctcagtcaccgtctctca

SEQ ID NO: 34 - DNA, 394 bp, Artificial Sequence
  12B4 mAb VL chain
atggattttcaggtgcagattttcagcttcctgctaatcagtgcctcagtcataatgtccagaggacaaattgttctcacccagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccgtacacgttcggaggggggaccaagctggaaataaaacgggctgatg

SEQ ID NO: 35 - DNA, 399 bp, Artificial Sequence
  24D12 mAb VH chain
atgggatggagctgtatcatgttctttttggtagcaacagctacaggtgtccactcccaggtccaactgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaaattactacggtactaatgtctggggcacagggaccacggtcaccgtctct

SEQ ID NO: 36 - DNA, 387 bp, Artificial Sequence
  24D12 mAb VL chain
atggagacagacacactgctgctatgggtactgctgctctgggttccaggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggaagcttacacgttcggaaggggggaccaaagctggaaa

SEQ ID NO: 37 - protein, 10 aa, Artificial Sequence
  VH CDR1
GYTFTSYWMH

SEQ ID NO: 38 - protein, 10 aa, Artificial Sequence
  VH CDR1
GYTFTNYWMH

SEQ ID NO: 39 - protein, 10 aa, Artificial Sequence
  VH CDR1
GYTFTSYWIT

SEQ ID NO: 40 - protein, 11 aa, Artificial Sequence
  VL CDR1
KASQNVGTNVA

SEQ ID NO: 41 - protein, 10 aa, Artificial Sequence
  VL CDR1
SASSSISYMH

SEQ ID NO: 42 - protein, 11 aa, Artificial Sequence
  VL CDR1
RASQEISGYLS

SEQ ID NO: 43 - protein, 16 aa, Artificial Sequence
  VL CDR1
RSSQSIVHSNGNTYLE

SEQ ID NO: 44 - protein, 10 aa, Artificial Sequence
  VL CDR1
SASSSVSYMH

SEQ ID NO: 45 - protein, 12 aa, Artificial Sequence
  VL CDR1
KSVSTSGYSYMH

SEQ ID NO: 46 - protein, 17 aa, Artificial Sequence
  VH CDR2
MIHPNSGSTNYNEKFKS

SEQ ID NO: 47 - protein, 17 aa, Artificial Sequence
  VH CDR2
DIYPGSGSTNYNEKFKS

SEQ ID NO: 48 - protein, 7 aa, Artificial Sequence
  VL CDR2
SASYRYS

SEQ ID NO: 49 - protein, 7 aa, Artificial Sequence
  VL CDR2
DTSKLAS

SEQ ID NO: 50 - protein, 7 aa, Artificial Sequence
  VL CDR2
AASTLDS

SEQ ID NO: 51 - protein, 7 aa, Artificial Sequence
  VL CDR2
KVSNRFS

SEQ ID NO: 52 - protein, 7 aa, Artificial Sequence
  VL CDR2
STSNLAS

SEQ ID NO: 53 - protein, 7 aa, Artificial Sequence
  VL CDR2
LVSNLES

SEQ ID NO: 54 - protein, 6 aa, Artificial Sequence
  VH CDR3
GGYFDY

SEQ ID NO: 55 - protein, 9 aa, Artificial Sequence
  VH CDR3
ARESRQLDY

SEQ ID NO: 56 - protein, 6 aa, Artificial Sequence
  VH CDR3
RGDGDY

SEQ ID NO: 57 - protein, 12 aa, Artificial Sequence
  VH CDR3
ARGGTRFSPFAY

SEQ ID NO: 58 - protein, 11 aa, Artificial Sequence
  VH CDR3
YYYGSSYAMDY

SEQ ID NO: 59 - protein, 6 aa, Artificial Sequence
  VH CDR3
YYGTNV

SEQ ID NO: 60 - protein, 9 aa, Artificial Sequence
  VL CDR3
QQYNSYPYT

SEQ ID NO: 61 - protein, 9 aa, Artificial Sequence
  VL CDR3
HQRSSYPLT

SEQ ID NO: 62 - protein, 9 aa, Artificial Sequence
  VL CDR3
LQYASYPYT

SEQ ID NO: 63 - protein, 9 aa, Artificial Sequence
  VL CDR3
FQGSHVPWT

SEQ ID NO: 64 - protein, 9 aa, Artificial Sequence
  VL CDR3
QQRSSYPYT

SEQ ID NO: 65 - protein, 8 aa, Artificial Sequence
  VL CDR3
QHIREAYT

SEQ ID NO: 66 - protein, 25 aa, Artificial Sequence
  VH FR1
QVQLQQPGAELVKPGASVKLSCKAS

SEQ ID NO: 67 - protein, 25 aa, Artificial Sequence [sequence omitted]

SEQ ID NO: 68 - protein, 23 aa, Artificial Sequence
  VL FR1
DIVMTQSQKFMSTSVGDRVSVTC

SEQ ID NO: 69 - protein, 23 aa, Artificial Sequence
  VL FR1
QIVLTQSPAIMSASPGEKVTMTC

SEQ ID NO: 70 - protein, 23 aa, Artificial Sequence
  VL FR1
DIQMTRHPSSLSASMGERVSLTC

SEQ ID NO: 71 - protein, 23 aa, Artificial Sequence
  VL FR1
DVLMTQTPLSLPVSLGDQASISC

SEQ ID NO: 72 - protein, 23 aa, Artificial Sequence
  VL FR1
QIVLTQSPAIMSASPGEKVTITC

SEQ ID NO: 73 - protein, 26 aa, Artificial Sequence
  VL FR1
DIVLTQSPASLAVSLGQRATISYRAS

SEQ ID NO: 74 - protein, 14 aa, Artificial Sequence
  VH FR2
WVKQRPGQGLEWIG

SEQ ID NO: 75 - protein, 15 aa, Artificial Sequence
  VL FR2
WYQQKPGQSPKALIY

SEQ ID NO: 76 - protein, 15 aa, Artificial Sequence
  VL FR2
WYQQKPGTSPKRWIY

SEQ ID NO: 77 - protein, 15 aa, Artificial Sequence
  VL FR2
WLQQKPDGTIKRLIY

SEQ ID NO: 78 - protein, 15 aa, Artificial Sequence
  VL FR2
WYLQKPGQSPKLLIY

SEQ ID NO: 79 - protein, 15 aa, Artificial Sequence
  VL FR2
WFQQKPGTSPKLWIY

SEQ ID NO: 80 - protein, 15 aa, Artificial Sequence
  VL FR2
WNQQKPGQPPRLLIY

SEQ ID NO: 81 - protein, 32 aa, Artificial Sequence
  VH FR3
KATLTVDKSSSTAYMQLSSLTSEDSAVYYCAR

SEQ ID NO: 82 - protein, 30 aa, Artificial Sequence
  VH FR3
KATLTVDKSSSTAYMQLSSLTSEDSAVYYC

SEQ ID NO: 83 - protein, 32 aa, Artificial Sequence
  VH FR3
KATLTVDKSSSTAYMQLSSLTSEDSAVYYCAN

SEQ ID NO: 84 - protein, 32 aa, Artificial Sequence
  VL FR3
GVPDRFTGSGSGTDFTLTISNVQSEDLAEYFC

SEQ ID NO: 85 - protein, 32 aa, Artificial Sequence
  VL FR3
GVPARFSGSGSGTSYSLTISSMEAEDAATYYC

SEQ ID NO: 86 - protein, 32 aa, Artificial Sequence
  VL FR3
GVPKRFSGSRSGSDYSLTISSLESEDFADYYC

SEQ ID NO: 87 - protein, 32 aa, Artificial Sequence
  VL FR3
GVPDRFSGSGSGTDFTLKISRVEAEDLGVYYC

SEQ ID NO: 88 - protein, 32 aa, Artificial Sequence
  VL FR3
GVPARFSGSGSGTSYSLTISRMEAEDAATYYC

SEQ ID NO: 89 - protein, 32 aa, Artificial Sequence
  VL FR3
GVPARFSGSGSGTDFTLNIHPVEEEDAATYYC

SEQ ID NO: 90 - protein, 11 aa, Artificial Sequence
  VH FR4
WGQGTTLTVSS

SEQ ID NO: 91 - protein, 12 aa, Artificial Sequence
  VH FR4
WGQGTLVTVSAA

SEQ ID NO: 92 - protein, 10 aa, Artificial Sequence
  VH FR4
WGQGTSVTVS

SEQ ID NO: 93 - protein, 10 aa, Artificial Sequence
  VH FR4
WGTGTTVTVS

SEQ ID NO: 94 - protein, 11 aa, Artificial Sequence
  VL FR4
FGGGTKLEIKR

SEQ ID NO: 95 - protein, 10 aa, Artificial Sequence
  VL FR4
FGAGTKLELK

SEQ ID NO: 96 - protein, 9 aa, Artificial Sequence
  VL FR4
FGGGTKLEI

SEQ ID NO: 97 - protein, 14 aa, Artificial Sequence
  VL FR4
FGGGTKLEIKRADA

SEQ ID NO: 98 - protein, 13 aa, Artificial Sequence
  VL FR4
FGGGTKLEIKRAD

SEQ ID NO: 99 - protein, 9 aa, Artificial Sequence
  VL FR4
FGRGDQSWK

SEQ ID NO: 100 - protein, 19 aa, Artificial Sequence
  Leader sequence
MGWSYIILFLVATATGVHS

SEQ ID NO: 101 - protein, 19 aa, Artificial Sequence
  Leader sequence
MGWSCIMLFLVATATGVHS

SEQ ID NO: 102 - protein, 19 aa, Artificial Sequence
  Leader sequence
MGWSCIMFFLVATATGVHS

SEQ ID NO: 103 - protein, 19 aa, Artificial Sequence
  Leader sequence
MEWSWVILILVAAATGVHS

SEQ ID NO: 104 - protein, 20 aa, Artificial Sequence
  Leader sequence
MKTQTQVFVYMLLWLSGVDG

SEQ ID NO: 105 - protein, 22 aa, Artificial Sequence
  Leader sequence
MDLQVQIFSFLLISASVILSRG

SEQ ID NO: 106 - protein, 15 aa, Artificial Sequence
  Leader sequence
MVLISLLFWFPGTRC

SEQ ID NO: 107 - protein, 19 aa, Artificial Sequence
  Leader sequence
MKMPVRLLVLMFWIPASSS

SEQ ID NO: 108 - protein, 22 aa, Artificial Sequence
  Leader sequence
MDFQVQIFSFLLISASVIMSRG

SEQ ID NO: 109 - protein, 20 aa, Artificial Sequence
  Leader sequence
METDTLLLWVLLLWVPGSTG

SEQ ID NO: 110 - protein, 115 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(98): FR3
  MISC_FEATURE (99)..(104): CDR3
  MISC_FEATURE (105)..(115): FR4
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARGGYFDYWGQGTTLTVSS

SEQ ID NO: 111 - protein, 116 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(96): FR3
  MISC_FEATURE (97)..(105): CDR3
  MISC_FEATURE (106)..(116): FR4
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARESRQLDYWGQGTTLTVSS

SEQ ID NO: 112 - protein, 115 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(98): FR3
  MISC_FEATURE (99)..(104): CDR3
  MISC_FEATURE (105)..(115): FR4
QVQLQQPGAELVKPGASVKLSCKASGYTFTNYWMHWVKQRPGQGLEWIGMIHPNSGSTNYNEKFKSKATLTVDKSSSTAYMQLSSLTSEDSAVYYCARRGDGDYWGQGTTLTVSS

SEQ ID NO: 113 - protein, 120 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(96): FR3
  MISC_FEATURE (97)..(108): CDR3
  MISC_FEATURE (109)..(120): FR4
QVQLQQPGAELVKPGASVKMSCKASGYTFTSYWITWVKQRPGQGLEWIGDIYPGSGSTNYNEKFKSKATLTVDTSSSTAYMQLSSLTSEDSAVYYCARGGTRFSPFAYWGQGTLVTVSAA

SEQ ID NO: 114 - protein, 119 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(98): FR3
  MISC_FEATURE (99)..(109): CDR3
  MISC_FEATURE (110)..(119): FR4
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARYYYGSSYAMDYWGQGTSVTVS

SEQ ID NO: 115 - protein, 114 aa, Artificial Sequence
  VH chain
  MISC_FEATURE (1)..(25): FR1
  MISC_FEATURE (26)..(35): CDR1
  MISC_FEATURE (36)..(49): FR2
  MISC_FEATURE (50)..(66): CDR2
  MISC_FEATURE (67)..(98): FR3
  MISC_FEATURE (99)..(104): CDR3
  MISC_FEATURE (105)..(114): FR4
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCANYYGTNVWGTGTTVTVS

SEQ ID NO: 116 - protein, 108 aa, Artificial Sequence
  VL chain
  MISC_FEATURE (1)..(23): FR1
  MISC_FEATURE (24)..(34): CDR1
  MISC_FEATURE (35)..(49): FR2
  MISC_FEATURE (50)..(56): CDR2
  MISC_FEATURE (57)..(88): FR3
  MISC_FEATURE (89)..(97): CDR3
  MISC_FEATURE (98)..(108): FR4
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIKR

SEQ ID NO: 117 - protein, 106 aa, Artificial Sequence
  VL chain
  MISC_FEATURE (1)..(23): FR1
  MISC_FEATURE (24)..(33): CDR1
  MISC_FEATURE (34)..(48): FR2
  MISC_FEATURE (49)..(55): CDR2
  MISC_FEATURE (56)..(87): FR3
  MISC_FEATURE (88)..(96): CDR3
  MISC_FEATURE (97)..(106): FR4
QIVLTQSPAIMSASPGEKVTMTCSASSSISYMHWYQQKPGTSPKRWIYDTSKLASGVPARFSGSGSGTSYSLTISSMEAEDAATYYCHQRSSYPLTFGAGTKLELK

SEQ ID NO: 118 - protein, 106 aa, Artificial Sequence
  VL chain
  MISC_FEATURE (1)..(23): FR1
  MISC_FEATURE (24)..(34): CDR1
  MISC_FEATURE (35)..(49): FR2
  MISC_FEATURE (50)..(56): CDR2
  MISC_FEATURE (57)..(88): FR3
  MISC_FEATURE (89)..(97): CDR3
  MISC_FEATURE (98)..(106): FR4
DIQMTRHPSSLSASMGERVSLTCRASQEISGYLSWLQQKPDGTIKRLIYAASTLDSGVPKRFSGSRSGSDYSLTISSLESEDFADYYCLQYASYPYTFGGGTKLEI

SEQ ID NO: 119 - protein, 116 aa, Artificial Sequence
  VL chain
  MISC_FEATURE (1)..(23): FR1
  MISC_FEATURE (24)..(39): CDR1
  MISC_FEATURE (40)..(54): FR2
  MISC_FEATURE (55)..(61): CDR2
  MISC_FEATURE (62)..(93): FR3
  MISC_FEATURE (94)..(102): CDR3
  MISC_FEATURE (103)..(116): FR4
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKRADA

SEQ ID NO: 120 - protein, 109 aa, Artificial Sequence
  VL chain
  MISC_FEATURE (1)..(23): FR1
  MISC_FEATURE (24)..(33): CDR1
  MISC_FEATURE (34)..(48): FR2
  MISC_FEATURE (49)..(55): CDR2
  MISC_FEATURE (56)..(87): FR3
  MISC_FEATURE (88)..(96): CDR3
  MISC_FEATURE (97)..(109): FR4
QIVLTQSPAIMSASPGEKVTITCSASSSVSYMHWFQQKPGTSPKLWIYSTSNLASGVPARFSGSGSGTSYSLTISRMEAEDAATYYCQQRSSYPYTFGGGTKLEIKRAD

SEQ ID NO: 121 - protein, 109 aa, Artificial Sequence
  VL chain
  MISC_FEATURE (1)..(26): FR1
  MISC_FEATURE (27)..(38): CDR1
  MISC_FEATURE (39)..(53): FR2
  MISC_FEATURE (54)..(60): CDR2
  MISC_FEATURE (61)..(92): FR3
  MISC_FEATURE (93)..(100): CDR3
  MISC_FEATURE (101)..(109): FR4
DIVLTQSPASLAVSLGQRATISYRASKSVSTSGYSYMHWNQQKPGQPPRLLIYLVSNLESGVPARFSGSGSGTDFTLNIHPVEEEDAATYYCQHIREAYTFGRGDQSWK